(19) United States
(12) Patent Application Publication
(10) Pub. No.: US 2022/0033489 A1
(43) Pub. Date: Feb. 3, 2022

(54) METHODS AND COMPOSITIONS FOR TREATING ASTHMA

(71) Applicant: THE CHILDREN'S MEDICAL CENTER CORPORATION, Boston, MA (US)

(72) Inventors: Talal Amine CHATILA, Belmont, MA (US); Hani HARB, Boston, MA (US); Brittany ESTY, Boston, MA (US); Wanda PHIPATANAKUL, Boston, MA (US); Lisa M. BARTNIKAS, Boston, MA (US)

(73) Assignee: THE CHILDREN'S MEDICAL CENTER CORPORATION, Boston, MA (US)

(21) Appl. No.: 17/299,664

(22) PCT Filed: Dec. 4, 2019
(86) PCT No.: PCT/US2019/064458

§ 371 Date: Jun. 3, 2021

Related U.S. Application Data
(60) Provisional application No. 62/775,127, filed on Dec. 4, 2018.

(51) Int. Cl. C07K 16/24; C12N 15/113; A61K 45/06; A61P 11/06

(52) U.S. Cl. CPC C07K 16/248; A61P 11/06; A61K 45/06; C12N 15/113

(57) ABSTRACT

Described herein are methods and compositions for treating asthma. Aspects of the invention relates to administering to a subject an agent that inhibits IL-6 signaling. Another aspect of the invention relates to administering the anti-IL-6R antibody, tocilizumab, to a subject in need thereof.

### CROSS-REFERENCE TO RELATED APPLICATION

This application is a 371 National Phase Entry of International Application No. PCT/US2019/064458, filed Dec. 4, 2019, which designated the U.S., and which claims the benefit under 35 U.S.C. § 119(e) of U.S. Provisional Application No. 62/775,127 filed on Dec. 4, 2018, the contents of which are incorporated herein by reference in their entireties.

### GOVERNMENT SUPPORT

This invention was made with Government support under Grant Nos. All 15699, AI065617, AI117983, AI126915, AI106822 and HL139124, awarded by the National Institutes of Health. The Government has certain rights in the invention.

### FIELD OF THE INVENTION

The field of the invention relates to the treatment of asthma.

### SEQUENCE LISTING

The instant application contains a Sequence Listing which has been submitted electronically in ASCII format and is hereby incorporated by reference in its entirety. Said ASCII copy, created on May 27, 2021, is named 701039-094130WOPT_SL.txt and is 9,088 bytes in size.

### BACKGROUND

Asthma is a chronic lung inflammatory disease with multiple phenotypic manifestations, underlined by several disease endotypes that reflect distinct pathophysiological mechanisms. A T helper cell type 2 high (T_{H}2^{High}) and mixed T_{H}2/T_{H}17 endotypes have been associated with severe asthma². The former is characterized by an eosinophilic and the latter by a mixed eosinophilic and neutrophilic airway inflammation, with the T_{H}2/T_{H}17 endotype manifesting as a difficult-to-control, steroid-resistant disease^{3,4}. Past research has demonstrated high sputum levels of IL-6 in patients with mixed eosinophilic/neutrophilic airway inflammation⁵. IL-6 blockade has been proposed as a treatment for asthma, however no such therapeutic is currently used to treat pediatric asthma⁶.

### SUMMARY

The present invention is based, in part, on the finding that patients presenting with severe, persistent atopic asthma showed a marked improvement when treated with an anti-IL-6 receptor agent, tocilizumab, as compared to other commonly used treatments for asthma. Tocilizumab is a humanized antibody that binds to, and inhibits, the IL-6 receptor. Accordingly, one aspect described herein provides a method of treating asthma, the method comprising administering to a subject in need thereof an effective amount of tocilizumab.

Another aspect described herein provides a method of treating asthma, the method comprising administering to a subject in need thereof an effective amount of an agent that inhibits IL-6 signaling.

In one embodiment of any aspect, the asthma is pediatric asthma, non-atopic asthma, and/or severe, persistent asthma.

In one embodiment of any aspect, the method further comprises, prior to administration, diagnosing a subject with having asthma.

In one embodiment of any aspect, the subject has a mutation in the IL4R gene.

In one embodiment of any aspect, the subject is a homozygous for IL4R dominant allele. In one embodiment of any aspect, the subject is a homozygous for IL4R mutant allele.

In one embodiment of any aspect, the method further comprises, prior to administration, identifying a subject as having a mutation in the IL4R gene.

In one embodiment of any aspect, the method further comprises, prior to administration, receiving results that identify a subject as having a mutation in the IL4R gene.

In one embodiment of any aspect, the agent targets IL-6 or IL-6 receptor (IL-6R).

In one embodiment of any aspect, the agent that inhibits IL-6 or IL-6R is selected from the group consisting of a small molecule, an antibody, a peptide, a genome editing system, an antisense oligonucleotide, and an RNAi (e.g., microRNA, siRNA, or shRNA).

In one embodiment of any aspect, the antibody is a humanized antibody. In one embodiment of any aspect, the humanized antibody is tocilizumab.

In one embodiment of any aspect, the expression level and/or activity of IL-6 or IL-6R is inhibited by at least 50%, at least 60%, at least 70%, at least 80%, at least 90%, or more as compared to an appropriate control.

In one embodiment of any aspect, administration decreases circulation of a cell selected from the group consisting of: a IL-4⁺Foxp3⁺ (T_{H}2-like) T regulatory cell, a IL-17⁺Foxp3⁺ (T_{H}17-like) T regulatory cell, a T_{H}2 cell, and a T_{H}17 cell. In one embodiment of any aspect, circulation is decreased by at least 50%, at least 60%, at least 70%, at least 80%, at least 90%, or more as compared to an appropriate control.

In one embodiment of any aspect, the agent or tocilizumab is administered at a concentration of 8 mg/kg or 10 mg/kg.

In one embodiment of any aspect, is the agent or tocilizumab administered once every 2 weeks or once every 4 weeks.

In one embodiment of any aspect, the method further comprises administering at least a second asthma therapeutic.

Another aspect described herein provides a composition comprising any of the agents that inhibit IL-6 signaling, as described herein. In one embodiment, the composition further comprises a pharmaceutically acceptable carrier.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with asthma. The term “treating” includes reducing or alleviating at least one adverse effect or symptom of an asthma (e.g., inflamed airway). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state or disorder (e.g., asthma) does not occur due to the actions of the methodology (such as, for example, administration of an agent that inhibits IL-6, or a composition described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not treated with a composition comprising a microbial consortium as described herein).

As used herein, the term “administering,” refers to the placement of a therapeutic (e.g., an agent that inhibits IL-6) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., asthma. A subject can be male or female. A subject can be a child (e.g., less than 18 years of age), or an adult (e.g., greater than 18 years of age).

A subject can be one who has been previously diagnosed with or identified as suffering from or having a disease or disorder in need of treatment (e.g., asthma) or one or more complications related to such a disease or disorder, and optionally, have already undergone treatment for the disease or disorder or the one or more complications related to the disease or disorder. Alternatively, a subject can also be one who has not been previously diagnosed as having such disease or disorder (e.g., asthma) or related complications. For example, a subject can be one who exhibits one or more risk factors for the disease or disorder or one or more complications related to the disease or disorder or a subject who does not exhibit risk factors.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit IL-6, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

Methods and compositions described herein require that the levels and/or activity of IL-6 are inhibited. As used herein, Interleukin-6 (IL-6), also known as CDF, HGF, HSF, BSF2, BSF-2, IFNB2, and IFN-beta-2 refers to a cytokine that functions in inflammation and the maturation of B cells. IL-6 is primarily produced at sites of acute and chronic inflammation, where it is secreted into the serum and induces a transcriptional inflammatory response through interleukin 6 receptor, alpha. IL-6 sequences are known for a number of species, e.g., human IL-6 (NCBI Gene ID: 3569) polypeptide (e.g., NCBI Ref Seq NP_000591.1) and mRNA (e.g., NCBI Ref Seq NM_000600.4). IL-6 can refer to human IL-6, including naturally occurring variants, molecules, and alleles thereof. IL-6 refers to the mammalian IL-6 of, e.g., mouse, rat, rabbit, dog, cat, cow, horse, pig, and the like. The nucleic sequence of SEQ ID NO: 1 comprises a nucleic sequence which encodes IL-6.

Methods and compositions described herein require that the levels and/or activity of Interleukin 6 receptor (IL-6R) are inhibited. As used herein, IL-6R, also known as IL6Q; gp80; CD126; IL6RA; IL6RQ; IL-6RA; IL-6R-1 refers to a cytokine that functions in inflammation and the maturation of B cells required to induce a transcriptional inflammatory response. IL-6R sequences are known for a number of species, e.g., human IL-6R (NCBI Gene ID: 3570) polypeptide (e.g., NCBI Ref Seq NP_000556.1) and mRNA (e.g., NCBI Ref Seq NM_000565.4). IL-6R can refer to human IL-6R, including naturally occurring variants, molecules, and alleles thereof. IL-6R refers to the mammalian IL-6R of, e.g., mouse, rat, rabbit, dog, cat, cow, horse, pig, and the like. The nucleic sequence of SEQ ID NO: 3 comprises a nucleic sequence which encodes IL-6R.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an asthma, or a biological sample that has not been contacted with an agent disclosed herein).

As used herein, an “appropriate control” refers to an untreated, otherwise identical cell or population (e.g., a patient who was not administered an agent or compositions described herein, or was administered by only a subset of agents described herein, as compared to a non-control cell).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1D present data that show analysis of cytokine production by T regulatory (Treg) cells in patient 1 and 2. FIGS. 1A and 1B, Flow cytometric analysis of IL-4 and IL-17 expression in Treg cells just prior to therapy and at 4, 8 and 10 months after tocilizumab treatment in patient 1 (FIG. 1A) and patient 2 (FIG. 1). FIGS. 1C and 1D, Graphical presentation of IL4 and IL-17 expression in Treg and T effector (Teff) cells in patient 1 (FIG. 1C) and patient 2 (FIG. 1D). *p<0.05, **<0.01, ***<0.001, ****<0.0001 by two-way ANOVA with Tukey post-test analysis.

FIGS. 2A and 2B present data that show analysis of cytokine production by T effector (Teff) cells in patient 1 and 2. FIGS. 2A and 2B, Flow cytometric analysis of IL-4 and IL-17 expression in Teff cells at baseline and at 4, 8 and 10 months after tocilizumab treatment in patient 1 (FIG. 2A) patient 2 (FIG. 2B).

FIGS. 3A-3I show Treg cell-specific deletion of Il6ra is partially protective against allergen and UFP-induced allergic airway inflammation. FIG. 3A, Representative PAS-stained sections of lung isolated from Foxp3YFPCre or Foxp3YFPCreNotch4Δ/Δ mice in PBS, OVA or OVA+UFP groups (200× magnification). FIG. 3B, Inflammation scores in the lung tissues isolated from the mouse groups described in FIG. 3A. FIG. 3C-3G, Airway hyper-responsiveness in response to methacholine (FIG. 3C), Total and OVA-specific IgE levels (FIG. 3D, 3E), absolute numbers of CD4+ T cells and eosinophils (FIGS. 3F and 3G). FIG. 3H-3I, absolute numbers of lung Foxp3+CD4+ T cells Treg cells (FIG. 3H) and lung Foxp3-CD4+ Teff cells secreting IL-4. IL-13, IL-17 or IFN-γ. Results are representative of 2 independent experiments. N=5 mice/group. p**<0.01, ***<0.001, ****<0.0001 by two-way ANOVA and post-test analysis.

FIGS. 4A and 4B show Treg cell-specific Il6ra deletion greatly attenuates the upregulation of Notch4 on lung Treg cells in allergic airway inflammation. FIG. 4A. Flow cytometric analysis of Notch4 expression on Treg cells isolated from the lungs of Foxp3^{YFPCre} and Foxp3^{YFPCre} Il6ra^{Δ/Δ} mice that were either sham sensitized or sensitized with OVA or OVA+UFP. FIG. 4B. Graphic representation of the percent Notch4 expression on lung Treg cells from the respectively indicated groups. N=5 mice/group. p****<0.0001 by two-way ANOVA and post-test analysis.

FIGS. 5A-5F show analysis of Notch4 expression on peripheral blood Treg and Teff cells in asthmatic patients and healthy controls. FIG. 5A. Flow cytometric analysis of Notch4 expression in CD4⁺CD25⁺Foxp3⁺ Treg cells in healthy subjects (control), mild persistent, moderate persistent and severe persistent asthmatic patients. FIG. 5B. Graphical presentation of Notch4 frequency and mean fluorescence intensity (MFI) in Treg cells. FIG. 5C. Flowcytometric analysis of Notch4 expression in CD4⁺CD25⁻Foxp3⁻ Teff cells in control, mild persistent, moderate persistent and severe persistent asthmatic patients. FIG. 5D. Graphical presentation of Notch4 frequency and mean fluorescence intensity (MFI) in Teff cells. FIG. 5E. In vitro suppression assay of anti-CD3 monoclonal antibody induced Teff cell proliferation using Notch4^{hi} versus Notch4^{lo} Treg cells from asthmatic subjects compares to healthy control Treg cells and Teff cells from a third-party healthy control. FIG. 5F. Notch4 expression in Treg cells of severe asthmatic patient 1 before and 3 months after Tocilizumab treatment.

FIGS. 6A-6D show in vitro induction of Notch4 expression on allergen-specific induced Treg (iTreg) cells. FIG. 6A. Naïve CD4⁺OT-II⁺ T Cells were co-cultured in vitro with cell-sorted alveolar macrophages that were pulsed with were either PBS or OVA₃₂₃₋₃₃₉ peptide (3 μM), either alone or together with UFP (10 μg). The cultures were otherwise left untreated or were further supplemented with recombinant mouse IL-6 (rIL-6; 10 ng/ml) (B), anti-IL-6 mAb (10 μg/ml), recombinant mouse IL-33 (rIL-33; 10 ng/ml) or the combination of rIL-6 and rIL-33. At the end of the co-cultures, CD4⁺Foxp3⁺ cells were gated and analyzed for Notch4 expression FIG. 6B. Graphical representation of Notch4 expression on Treg cells with different stimuli. FIG. 6C. naïve T-cell differentiation into Treg cells in Foxp3^{YFPCre}, Foxp3^{YFPCre} Il6ra^{Δ/Δ} or Foxp3^{YFPCre} Stat3^{Δ/Δ} mice with stimulation with recombinant IL-6 (rIL-6; 10 ng/ml) FIG. 6D. Chromatin Immunoprecipitation (ChIP) of mock control (IgG) or STAT3 for Foxp3^{YFPCre} or Foxp3^{YFPCre} Stat3^{Δ/Δ} Treg cell with and without IL-6 stimulation. Results represent means of 3-5 replicate cultures/samples, each derived from one mouse, 1 S.E.M. *P<0.05, ***P<0.001 and ****P<0.0001 by One-way ANOVA with posttest analysis.

### DETAILED DESCRIPTION

### Treating or Preventing Asthma

Research has demonstrated that high sputum levels of IL-6 is associated with patients with mixed eosinophilic/neutrophilic airway inflammation 5. Thus, IL-6 blockade has been proposed as a treatment for asthma, though no such therapeutic exists for pediatric asthma⁶.: The IL-4 receptor alpha chain variant R576 (IL-4Ra-R576) promotes mixed T_{H}2/T_{H}17 airway inflammation^{7,8}. Described herein is the response of two patients with severe persistent, non-atopic asthma with evidence of T_{H}2/T_{H}17 inflammation treated with tocilizumab, a humanized anti-IL-6 receptor (IL-6R) mAb.

One aspect of the invention is a method of treating asthma by administering to a subject in need thereof an agent that inhibits IL-6 signaling. Il-6 signaling can be inhibited via directly or indirectly inhibiting IL-6 or IL-6 receptor (IL-6R). Agents that target each component are identified herein below. Another aspect provided herein is a method of treating asthma by administering to a subject in need thereof tocilizumab.

In one embodiment, an agent that inhibits IL-6 or IL-6R is administered as a prophalytic treatment to prevent asthma in a subject at risk of developing asthma, for example, severe persistent asthma. Risk factors for developing asthma, e.g., severe persistent asthma, are described herein below.

As used herein, an “asthma” refers to a disease characterized by inflammation in the airways of the lungs, reversible airways obstructions, bronchospasms, wheezing, coughing, tightness of the chest, and shortness of breath. Asthma is thought to be caused by environmental and genetic factors, include, but not limited to exposure to air pollutants and allergens, aspirin and beta blockers, and a family history of asthma. In one embodiment, the asthma is pediatric asthma (also known as childhood asthma). As used herein, “pediatric asthma” refers to a diagnoses of asthma in a subject under the age of 18 years old.

Asthma is classified by the frequency of symptoms, the severity of symptoms, forced expiratory volume in one second (FEV1), and peak expiratory flow rate. Asthma can further be classified based on the subject's response to a medication, e.g., atopic or non-atopic, wherein atropic refers to a predisposition towards developing a type 1 hypersensitivity. Asthma can be classified as intermittent, mild, moderate, or severe.

Asthma is considered intermittent if, for example, without treatment any of the following are true: (1) Symptoms occur two days or less per week and do not interfere with normal activities; (2) Nighttime symptoms occur two days or less per month; (3) When not having an asthma attack, lung function tests are normal (at 80 percent or more of the expected value); and (4) vary little from morning to afternoon.

Asthma is considered mild persistent if, for example, without treatment any of the following are true: (1) Symptoms occur more than two days a week, but not every day, and nighttime symptoms occur three to four times a month; (2) Asthma attacks interfere with normal daily activities; (3) Lung function tests are normal when not having an attack (tests are 80 percent or more of the expected value); and (4) may vary a small amount from morning to afternoon.

Asthma is considered moderate persistent if, for example, without treatment any of the following are true: (1) A daily occurrence of symptoms and a short-acting inhaler is used every day; (2) Symptoms interfere with daily activities; Nighttime symptoms occur more than one time a week, but do not happen every day; and (4) Lung function tests are abnormal and varies more than 30 percent from morning to afternoon.

In one embodiment, the asthma is severe asthma. As used herein, “severe asthma” refers to asthma with increased severity that is unresponsive to routine therapy and that, if severe enough, can lead to death. In another embodiment, the asthma can be acute severe asthma. As used herein, “acute severe asthma” refers to an asthmatic attack that presents with increased severity that is unresponsive to routine therapy and can lead to death.

In one embodiment with severe persistent asthma. Asthma is considered severe persistent if, for example, without treatment any of the following are true: (1) Symptoms occur throughout each day and severely limit daily physical activities; (2) Nighttime symptoms occur often, sometimes every night; and (3) Lung function tests are abnormal and may vary greatly from morning to afternoon. Current treatment of severe persistent asthma include, but are not limited to long-term control medicines (inhaled corticosteroids) that reduce inflammation of the airways to prevent asthma symptoms and asthma attacks; long-acting bronchodilators and a quick-relief medicine (short-acting beta agonist or bronchodilator), and anti-inflammatory medicines known as “leukotriene modifiers.”

In various embodiments, the asthma is allergic asthma (e.g., induced by exposure to allergens), asthma without allergies (e.g., induced by an upper respiratory infection, such as a cold, flu, or rhinovirus), aspirin exacerbated respiratory disease (e.g., induced by the intake of aspirin), exercised-induced asthma, cough variant (e.g., characterized by a dry, hacking cough), or occupational asthma (e.g., induced by an irritant a subject is exposed to on a job, for example, a fire fighter is exposed to smoke, and can experience smoke-inhalation, while performing their job). A skilled clinician can identify a type of asthma a subject has, or is at risk of having (e.g., a fire fighter would be at risk of having occupational asthma), using standard techniques.

A subject can be identified as having or be at risk of having asthma by a skilled clinician. Diagnostic tests useful in identifying a subject having asthma are known in the art, and further described herein below.

As used herein a subject “at risk of having asthma” refers to a subject who is in contact, or potentially in contact, with known asthma triggers (e.g., factors that can result in the onset of asthma). Non-limiting factors that can, e.g., trigger the onset of asthma, include airborne substances, (e.g., pollen, dust mites, mold spores, pet dander or particles of cockroach waste); respiratory infections, (e.g., the common cold); physical activity (e.g., can trigger exercised-induced asthma); cold air; air pollutants and irritants, (e.g., smoke and cigarette smoke); certain medications (e.g., blockers, aspirin, ibuprofen (Advil, Motrin IB, others) and naproxen (Aleve)); strong emotions or stress; sulfites and preservatives added food and/or beverages (e.g., found in shrimp, dried fruit, processed potatoes, beer, and wine); and gastroesophageal reflux disease (GERD). A subject is also considered at risk of asthma if the subject has a family history of asthma (e.g., if an immediate family member has had asthma).

In one embodiment, the subject has a mutation in the IL4R gene. In various embodiments, the subject has is homozygous for IL4R dominant allele or homozygous for IL4R mutant allele. Mutations in the IL4R gene, for example, the IL4RA^{R576} mutation, is associated with increased severity of asthma.

In one embodiment, the method of treating asthma is a subject in need thereof further comprising, prior to administration, identifying a subject as having a mutation in the IL4R gene. In another embodiment, the method of treating asthma is a subject in need thereof further comprising, prior to administration, receiving results that identify a subject as having a mutation in the IL4R gene. Methods (i.e., assays) for identifying a mutation in a gene (e.g., the IL4R gene) include genome sequence or PCR-based screening of, for example, a biological sample obtained from the subject. A biological sample can be, for example, a blood sample, a sputum sample, or a tissue sample.

### Agents

In one aspect, an agent that inhibits IL-6 or IL-6R is administered to a subject having, or at risk of having asthma. In one embodiment, the agent that inhibits IL-6 or IL-6R is a small molecule, an antibody or antibody fragment, a peptide, an antisense oligonucleotide, a genome editing system, or an RNAi.

In one embodiment, the agent inhibits IL-6R-mediated signaling. As described herein, “inhibiting IL-6 mediated signaling” refer to the inhibition of any of the key components of this pathway, for example IL-6 or IL-6R, that reduces or inhibits the level or activity of IL-6 signaling. In one embodiment, the agent described herein can inhibit the level or activity of IL-6 mediated signaling, for example, by inhibiting a key component of IL-6 mediated signaling, e.g., IL-6 or IL-6R. This inhibition can be direct, for example by directly inhibiting a component of the IL-6 mediated signaling pathway, for example IL-6 or IL-6R. In an alternate embodiment, the agent can indirectly inhibit a component of the IL-6 mediated signaling pathway, for example via inhibition of a IL-6 or IL-6R regulator.

An agent is considered effective for inhibiting IL-6 or IL-6R if, for example, upon administration, it inhibits the presence, amount, activity and/or level of IL-6 or IL-6R in the cell.

In one embodiment, inhibiting IL-6 or IL-6R decreases circulation of a IL-4⁺Foxp3⁺ (T_{H}2-like) T regulatory cell, a IL-17⁺Foxp3⁺ (T_{H}17-like) T regulatory cell, a T_{H}2 cell, or a T_{H}17 cell. In one embodiment, circulation is reduced by at least 5%, 10%, 15%, 20%, 25%, 30%, 35%, 40%, 45%, 50%, 55%, 60%, 65%, 70%, 75%, 80%, 85%, 90% 95%, 99%, or more. A skilled practitioner will be able to determine if a IL-4⁺Foxp3⁺ (T_{H}2-like) T regulatory cell, a IL-17⁺Foxp3⁺ (T_{H}17-like) T regulatory cell, a T_{H}2 cell, or a T_{H}17 cell is decreased in circulation following administration of an agent that inhibits IL-6 mediated signaling.

An agent can inhibit e.g., the transcription, or the translation of IL-6 or IL-6R in the cell. An agent can inhibit the activity or alter the activity (e.g., such that the activity no longer occurs, or occurs at a reduced rate) of IL-6 or IL-6R in the cell (e.g., IL-6 or IL-6R's expression).

In one embodiment, an agent that inhibits IL-6 or IL-6R inhibits the expression level or activity of IL-6 or IL-6R. To determine is an agent is effective at inhibiting IL-6 or IL-6R, mRNA and protein levels of a given target (e.g., IL-6 or IL-6R) can be assessed using RT-PCR and western-blotting, respectively. Biological assays that detect the activity of IL-6 or IL-6R can be used to assess if a decrease in circulation of a IL-4⁺Foxp3⁺ (T_{H}2-like) T regulatory cell, a IL-17⁺Foxp3⁺ (T_{H}17-like) T regulatory cell, a T_{H}2 cell, or a T_{H}17 cell has occurred.

In one embodiment, an agent that inhibits the level and/or activity of IL-6 or IL-6R by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the level and/or activity of IL-6 prior to administration of the agent, or the level and/or activity of IL-6 in a population of cells that was not in contact with the agent.

The agent may function directly in the form in which it is administered. Alternatively, the agent can be modified or utilized intracellularly to produce something which inhibits IL-6 or IL-6R, such as introduction of a nucleic acid sequence into the cell and its transcription resulting in the production of the nucleic acid and/or protein inhibitor of IL-6 or IL-6R. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits IL-6 or IL-6R. Methods for screening small molecules are known in the art and can be used to identify a small molecule that is efficient at, for example, decreasing circulation of a IL-4⁺Foxp3⁺ (T_{H}2-like) T regulatory cell, a IL-17⁺Foxp3⁺ (T_{H}17-like) T regulatory cell, a T_{H}2 cell, or a T_{H}17 cell, given the desired target (e.g., IL-6 or IL-6R).

In various embodiments, the agent that inhibits IL-6 or IL-6R is an antibody or antigen-binding fragment thereof, or an antibody reagent that is specific for IL-6 or IL-6R. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the agent that inhibits IL-6 or IL-6R is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use. Methods for humanizing a non-human antibody are known in the art.

In one embodiment, the antibody or antibody reagent binds to an amino acid sequence that corresponds to the amino acid sequence encoding IL-6 (SEQ ID NO: 2).

(SEQ ID NO: 2)
MNSFSTSAFGPVAFSLGLLLVLPAAFPAPVPPGEDSKDVAAPHRQPLTSS
ERIDKQIRYILDGISALRKETCNKSNMCESSKEALAENNLNLPKMAEKDG
CFQSGFNEETCLVKIITGLLEFEVYLEYLQNRFESSEEQARAVQMSTKVL
IQFLQKKAKNLDAITTPDPTTNASLLTKLQAQNQWLQDMTTHLILRSFKE
FLQSSLRALRQM

In another embodiment, the anti-IL-6 antibody or antibody reagent binds to an amino acid sequence that comprises the sequence of SEQ ID NO: 2; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-IL-6 antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., IL-6, and for example, decrease circulation of a IL-4⁺Foxp3⁺ (T_{H}2-like) T regulatory cell, a IL-17⁺Foxp3⁺ (T_{H}17-like) T regulatory cell, a T_{H}2 cell, or a T_{H}17 cell.

In one embodiment, the antibody or antibody reagent binds to an amino acid sequence that corresponds to the amino acid sequence encoding IL-6R (SEQ ID NO: 4).

(SEQ ID NO: 4)
MLAVGCALLAALLAAPGAALAPRRCPAQEVARGVLTSLPGDSVTLTCPGV
EPEDNATVHWVLRKPAAGSHPSRWAGMGRRLLLRSVQLHDSGNYSCYRAG
RPAGTVHLLVDVPPEEPQLSCFRKSPLSNVVCEWGPRSTPSLTTKAVLLV
RKFQNSPAEDFQEPCQYSQESQKFSCQLAVPEGDSSFYIVSMCVASSVGS
KFSKTQTFQGCGILQPDPPANTTVTAVARNPRWLSVTWQDPHSWNSSFYR
LRFELRYRAERSKTFTTWMVKDLQHHCVIHDAWSGLRHVVQLRAQEEFGQ
GEWSEWSPEAMGTPWTESRSPPAENEVSTPMQALTTNKDDDNILFRDSAN
ATSLPVQDSSSVPLPTFLVAGGSLAFGTLLCIAIVLRFKKTWKLRALKEG
KTSMHPPYSLGQLVPERPRPTPVLVPLISPPVSPSSLGSDNTSSHNRPDA
RDPRSPYDISNTDYFFPR

In another embodiment, the anti-IL-6R antibody or antibody reagent binds to an amino acid sequence that comprises the sequence of SEQ ID NO: 4; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 4. In one embodiment, the anti-IL-6R antibody or antibody reagent binds to an amino acid sequence that comprises the entire sequence of SEQ ID NO: 4. In another embodiment, the antibody or antibody reagent binds to an amino acid sequence that comprises a fragment of the sequence of SEQ ID NO: 4, wherein the fragment is sufficient to bind its target, e.g., IL-6R, and for example, decrease circulation of a IL-4⁺Foxp3⁺ (T_{H}2-like) T regulatory cell, a IL-17⁺Foxp3⁺ (T_{H}17-like) T regulatory cell, a T_{H}2 cell, or a T_{H}17 cell.

Exemplary anti-IL-6 antibodies are further described, for example, in U.S. Pat. Nos. 8,562,992; 10,093,732; 10,040,851; 9,951,136; 9,879,074; 9,340,613; 8,846,037; 7,955,597; 7,919,095; 7,291,721; and 5,559,012, the contents of which are incorporated herein by reference in their entireties.

In one embodiment, the anti-IL-6R antibody is Tocilizumab (RoActemra®; Chugai/Roche), a first-in-class humanized monoclonal antibody that binds specifically to both sIL-6R and mIL-6R and inhibits IL-6R-mediated signaling. Tocilizumab has been approved for patients with moderate to severe rheumatoid arthritis unresponsive to available DMARDs. Tocilizumab is further described in, for example, U.S. Pat. No. 10,105,418; and Published US Application Nos US20110245473 and US20120253016, which the contents of which are incorporated herein by reference in their entireties.

In one embodiment, the agent that inhibits IL-6 or IL-6R is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., IL-6 or IL-6R. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits IL-6 may comprise at least 5, at least 10, at least 15, at least 20, at least 25, at least 30, or more bases complementary to a portion of the coding sequence of the human IL-6 gene (e.g., SEQ ID NO: 1).

SEQ ID NO: 1 is a nucleotide sequence that encodes IL-6.

(SEQ ID NO: 1)
tatgaactcc ttctccacaa gcgccttcgg tccagttgcc
ttctccctgg ggctgctcct ggtgttgcct gctgccttcc
ctgccccagt acccccagga gaagattcca aagatgtagc
cgccccacac agacagccac tcacctcttc agaacgaatt
gacaaacaaa ttcggtacat cctcgacggc atctcagccc
tgagaaagga gacatgtaac aagagtaaca tgtgtgaaag
cagcaaagag gcactggcag aaaacaacct gaaccttcca
aagatggctg aaaaagatgg atgcttccaa tctggattca
atgaggagac ttgcctggtg aaaatcatca ctggtctttt
ggagtttgag gtatacctag agtacctcca gaacagattt
gagagtagtg aggaacaagc cagagctgtg cagatgagta
caaaagtcct gatccagttc ctgcagaaaa aggcaaagaa
tctagatgca ataaccaccc ctgacccaac cacaaatgcc
agcctgctga cgaagctgca ggcacagaac cagtggctgc
aggacatgac aactcatctc attctgcgca gctttaagga
gttcctgcag tccagcctga gggctcttcg gcaaatgtag

In another example, an antisense oligonucleotide that inhibits IL-6R may comprise at least 5, at least 10, at least 15, at least 20, at least 25, at least 30, or more bases complementary to a portion of the coding sequence of the human IL-6R gene (e.g., SEQ ID NO: 3).

SEQ ID NO: 3 is a nucleotide sequence that encodes IL-6R.

| (SEQ ID NO: 3) | |
|---|---|
| | atg ctggccgtcg |
| 301 | gctgcgcgct gctggctgcc ctgctggccg cgccgggagc ggcgctggcc ccaaggcgct |
| 361 | gccctgcgca ggaggtggcg agaggcgtgc tgaccagtct gccaggagac agcgtgactc |
| 421 | tgacctgccc gggggtagag ccggaagaca atgccactgt tcactgggtg ctcaggaagc |
| 481 | cggctgcagg ctcccacccc agcagatggg ctggcatggg aaggaggctg ctgctgaggt |
| 541 | cggtgcagct ccacgactct ggaaactatt catgctaccg ggccggccgc ccagctggga |
| 601 | ctgtgcactt gctggtggat gttccccccg aggagcccca gctctcctgc ttccggaaga |
| 661 | gccccctcag caatgttgtt tgtgagtggg gtcctcggag caccccatcc ctgacgacaa |
| 721 | aggctgtgct cttggtgagg aagtttcaga acagtccggc cgaagacttc caggagccgt |
| 781 | gccagtattc ccaggagtcc cagaagttct cctgccagtt agcagtcccg gagggagaca |
| 841 | gctctttcta catagtgtcc atgtgcgtcg ccagtagtgt cgggagcaag ttcagcaaaa |
| 901 | ctcaaacctt tcagggttgt ggaatcttgc agcctgatcc gcctgccaac atcacagtca |
| 961 | ctgccgtggc cagaaacccc cgctggctca gtgtcacctg gcaagacccc cactcctgga |
| 1021 | actcatcttt ctacagacta cggtttgagc tcagatatcg ggctgaacgg tcaaagacat |
| 1081 | tcacaacatg gatggtcaag gacctccagc atcactgtgt catccacgac gcctggagcg |
| 1141 | gcctgaggca cgtggtgcag cttcgtgccc aggaggagtt cgggcaaggc gagtggagcg |
| 1201 | agtggagccc ggaggccatg ggcacgcctt ggacagaatc caggagtcct ccagctgaga |
| 1261 | acgaggtgtc cacccccatg caggcactta ctactaataa agacgatgat aatattctct |
| 1321 | tcagagattc tgcaaatgcg acaagcctcc cagtgcaaga ttcttcttca gtaccactgc |
| 1381 | ccacattcct ggttgctgga gggagcctgg ccttcggaac gctcctctgc attgccattg |
| 1441 | ttctgaggtt caagaagacg tggaagctgc gggctctgaa ggaaggcaag acaagcatgc |
| 1501 | atccgccgta ctctttgggg cagctggtcc cggagaggcc tcgacccacc ccagtgcttg |
| 1561 | ttcctctcat ctccccaccg gtgtccccca gcagcctggg gtctgacaat acctcgagcc |
| 1621 | acaaccgacc agatgccagg gacccacgga gcccttatga catcagcaat acagactact |
| 1681 | tcttccccag atag |

In one embodiment, IL-6 or IL-6R is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits IL-6 or IL-6R by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. IL-6. In some embodiments of any of the aspects, the agent is siRNA that inhibits IL-6. In some embodiments of any of the aspects, the agent is shRNA that inhibits IL-6.

One skilled in the art would be able to design siRNA, shRNA, or miRNA to target IL-6, e.g., using publically available design tools. siRNA, shRNA, or miRNA is commonly made using companies such as Dharmacon (Layfayette, Colo.) or Sigma Aldrich (St. Louis, Mo.).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits IL-6 or IL-6R. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., IL-6 or IL-6R), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., IL-6 or IL-6R, for its downregulation, for example by transfecting the siRNA, shRNA, or miRNA into cells and detecting the levels of a gene or gene product (e.g., IL-6 or IL-6R) found within the cell via PCR-based assay or western-blotting, respectively.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., an IL-6 or IL-6R inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

One aspect provided herein is a composition comprising any of the agents that inhibits IL-6-mediated signaling, as described herein.

In one embodiment, the composition further comprises a pharmaceutically acceptable carrier. As used herein, the term “pharmaceutically acceptable”, and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. Each carrier must also be “acceptable” in the sense of being compatible with the other ingredients of the formulation. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. The pharmaceutical formulation contains a compound of the invention in combination with one or more pharmaceutically acceptable ingredients. The carrier can be in the form of a solid, semi-solid or liquid diluent, cream or a capsule. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition will depend on the nature of the disorder or condition, and can be determined by standard clinical techniques. The phrase “pharmaceutically acceptable carrier or diluent” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body.

Compositions described herein can be formulated for any route of administration described herein below. Methods for formulating a composition for a desired administration are further discussed below.

### Administration

In some embodiments, the methods described herein relate to treating a subject having or diagnosed as having an asthma disease comprising administering an agent that inhibits IL-6-mediated signaling (e.g., via inhibition of IL-6 or IL-6R) as described herein. Subjects having an asthma can be identified by a physician using current methods (i.e. assays) of diagnosing a condition. Symptoms and/or complications of asthma, which characterize these disease and aid in diagnosis are well known in the art and include but are not limited to, persistent cough, trouble breathing, wheezing, and shortness of breath. Tests that may aid in a diagnosis of, e.g. asthma, include but are not limited methacholine challenge, nitric oxide test, allergy testing, and sputum eosinophils. A family history of, e.g., asthma, will also aid in determining if a subject is likely to have the condition or in making a diagnosis of asthma.

The agents described herein (e.g., an agent that inhibits IL-6 signaling, e.g., via inhibition of IL-6 or IL-6R) can be administered to a subject having or diagnosed as having asthma. In some embodiments, the methods described herein comprise administering an effective amount of an agent to a subject in order to alleviate at least one symptom of, e.g., asthma. As used herein, “alleviating at least one symptom of asthma” is ameliorating any condition or symptom associated with, e.g., asthma (e.g., persistent cough, trouble breathing, wheezing, and shortness of breath). As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. In one embodiment, the agent is administered systemically or locally (e.g., to the lungs). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner.

In one embodiment, the agent, or compositions comprising an agent is administered through inhalation. Thus, in one embodiment, a composition comprising an agent described herein is formulated for aerosol delivery.

The term “effective amount” as used herein refers to the amount of an agent (e.g., an agent that inhibits IL-6 signaling, e.g., via inhibition of IL-6 or IL-6R) can be administered to a subject having or diagnosed as having asthma needed to alleviate at least one or more symptom of, e.g., asthma. The term “therapeutically effective amount” therefore refers to an amount of an agent that is sufficient to provide, e.g., a particular anti-asthma effect when administered to a typical subject. An effective amount as used herein, in various contexts, would also include an amount of an agent sufficient to delay the development of a symptom of, e.g., asthma, alter the course of a symptom of, e.g., asthma (e.g., slowing the progression of loss of lung function, inappropriate breathing, or wheezing), or reverse a symptom of, e.g., (e.g., improve lung function or breathing). Thus, it is not generally practicable to specify an exact “effective amount”. However, for any given case, an appropriate “effective amount” can be determined by one of ordinary skill in the art using only routine experimentation.

In one embodiment, the agent is administered continuously (e.g., at constant levels over a period of time). Continuous administration of an agent can be achieved, e.g., by epidermal patches, continuous release formulations, or on-body injectors.

In one embodiment, the agent, for example Tocilizumab, is agent is administered at a concentration of 8 mg/kg or 10 mg/kg. In another embodiment, the agent is administered at a concentration of 1 mg/kg-10 mg/kg, 2 mg/kg-10 mg/kg, 3 mg/kg-10 mg/kg, 4 mg/kg-10 mg/kg, 5 mg/kg-10 mg/kg, 6 mg/kg-10 mg/kg, 7 mg/kg-10 mg/kg, 8 mg/kg-10 mg/kg, 9 mg/kg-10 mg/kg, 1 mg/kg-9 mg/kg, 1 mg/kg-8 mg/kg, 1 mg/kg-7 mg/kg, 1 mg/kg-6 mg/kg, 1 mg/kg-5 mg/kg, 1 mg/kg-4 mg/kg, 1 mg/kg-3 mg/kg, 1 mg/kg-2 mg/kg, 2.5 mg/kg-10 mg/kg, 5 mg/kg-10 mg/kg, 7.5 mg/kg-10 mg/kg, 1 mg/kg-7.5 mg/kg, or 1 mg/kg-2.5 mg/kg. In one embodiment, the agent is administered at a concentration greater than 10 mg/kg.

In one embodiment, the agent, for example Tocilizumab, is administered once every 2 weeks or once every 4 weeks. An agent described herein can be administered at least once a day, a week, every 3 weeks, a month, every 2 months, every 3 months, every 4 months, every 5 months, every 6 months, every 7 months, every 8 months, every 9 months, every 10 months, every 11 months, a year, or more. In a preferred embodiment, Tocilizumab is administered at least once a month.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combinational Therapy

In one embodiment, the agent described herein is used as a monotherapy. In one embodiment, the agents described herein can be used in combination with other known agents and therapies for asthma. Administered “in combination,” as used herein, means that two (or more) different treatments are delivered to the subject during the course of the subject's affliction with the disorder, e.g., the two or more treatments are delivered after the subject has been diagnosed with the disorder or disease (for example, asthma) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Exemplary therapeutics used to treat asthma include, but are not limited to, inhaled corticosteroids (e.g., fluticasone (Flonase, Flovent HFA), budesonide (Pulmicort Flexhaler, Rhinocort), flunisolide (Aerospan HFA), ciclesonide (Alvesco, Omnaris, Zetonna), beclomethasone (Qnasl, Qvar), mometasone (Asmanex) and leukotriene modifiers (e.g., montelukast (Singulair), zafirlukast (Accolate) and zileuton (Zyflo)); long-acting beta agonists (e.g., salmeterol (Serevent) and formoterol (Foradil, Perforomist)); combination inhalers (e.g., fluticasone-salmeterol (Advair Diskus), budesonide-formoterol (Symbicort) and formoterol-mometasone (Dulera)); theophylline (e.g., Theophylline (Theo-24, Elixophylline)); short-acting beta agonists (e.g., albuterol (ProAir HFA, Ventolin HFA, others) and levalbuterol (Xopenex)); ipratropium (e.g., Atrovent); and oral and intravenous corticosteroids.

When administered in combination, the agent and the at least one additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of asthma) is lower (e.g., at least 20%, at least 30%, at least 40%, or at least 50% lower) than the amount or dosage of each agent individually required to achieve the same therapeutic effect.

### Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Aerosol Formulations

An agent that inhibits IL-6 signaling or composition comprising an agent that inhibits IL-6 signaling (e.g., via inhibition of a component of IL-6 signaling) can be administered directly to the airways of a subject in the form of an aerosol or by nebulization. For use as aerosols, an agent that inhibits IL-6 signaling in solution or suspension may be packaged in a pressurized aerosol container together with suitable propellants, for example, hydrocarbon propellants like propane, butane, or isobutane with conventional adjuvants. An agent that inhibits IL-6 signaling can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device.

As is well known, any suitable gas can be used to apply pressure during the nebulization, with preferred gases to date being those which are chemically inert to a modulator of an agent that inhibits IL-6 signaling. Exemplary gases including, but are not limited to, nitrogen, argon or helium can be used to high advantage.

In some embodiments, an agent that inhibits IL-6 signaling can also be administered directly to the airways in the form of a dry powder. For use as a dry powder, a GHK tripeptide can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The correct dosage of the composition is delivered to the patient.

A dry powder inhaler (i.e. Turbuhaler (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5p m. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of an agent that inhibits IL-6 signaling thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

### Controlled and Delayed Release Dosage Forms

In some embodiments of the aspects described herein, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agents described herein, e.g., for the treatment of an asthma, can be determined by the skilled practitioner. However, a treatment is considered “effective treatment,” as the term is used herein, if one or more of the signs or symptoms of, e.g., asthma, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., decreased airway inflammation, increased lung function, restored normal breathing. Efficacy can also be measured by a failure of an individual to worsen as assessed by hospitalization, or need for medical interventions (i.e., progression of diminished lung function, complications with breathing, asthmatic attack frequencies). Methods of measuring these indicators are known to those of skill in the art and/or are described herein.

Efficacy can be assessed in animal models of a condition described herein, for example, a mouse model or an appropriate animal model of asthma, as the case may be. When using an experimental animal model, efficacy of treatment is evidenced when a statistically significant change in a marker is observed, e.g., decreased airway inflammation, increased lung function, restored normal breathing.

Efficacy of an agent that inhibits IL-6 signaling can additionally be assessed using methods described herein.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention provided herein can be further described in any of the following numbered paragraphs.
1) A method of treating asthma, the method comprising administering to a subject in need thereof an effective amount of an agent that inhibits the IL-6 signaling.
2) The method of paragraph 1, wherein the asthma is pediatric asthma.
3) The method of any of the preceding paragraphs, wherein the asthma is non-atopic asthma.
4) The method of any of the preceding paragraphs, wherein the asthma is severe, persistent asthma.
5) The method of any of the preceding paragraphs, further comprising, prior to administration, diagnosing a subject with having asthma.
6) The method of any of the preceding paragraphs, wherein the subject has a mutation in the IL4R gene.
7) The method of any of the preceding paragraphs, wherein the subject is a homozygous for TL4R dominant allele.
8) The method of any of the preceding paragraphs, wherein the subject is a homozygous for IL4R mutant allele.
9) The method of any of the preceding paragraphs, further comprising, prior to administration, identifying a subject as having a mutation in the IL4R gene.
10) The method of any of the preceding paragraphs, further comprising, prior to administration, receiving results that identify a subject as having a mutation in the IL4R gene.
11) The method of any of the preceding paragraphs, wherein the agent that inhibits IL-6 signaling is selected from the group consisting of a small molecule, an antibody, a peptide, a genome editing system, an antisense oligonucleotide, and an RNAi.
12) The method of any of the preceding paragraphs, wherein the RNAi is a microRNA, an siRNA, or a shRNA.
13) The method of any of the preceding paragraphs, wherein the antibody is a humanized antibody.
14) The method of any of the preceding paragraphs, wherein the humanized antibody is tocilizumab.
15) The method of any of the preceding paragraphs, wherein the agent targets IL-6 or IL-6 receptor (IL-6R).
16) The method of any of the preceding paragraphs, wherein inhibiting IL-6 or IL-6R is inhibiting the expression level and/or activity of IL-6 or IL-6R.
17) The method of any of the preceding paragraphs, wherein the expression level and/or activity of IL-6 or IL-6R is inhibited by at least 50%, at least 60%, at least 70%, at least 80%, at least 90%, or more as compared to an appropriate control.
18) The method of any of the preceding paragraphs, wherein administration decreases circulation of a cell selected from the group consisting of: a IL-4⁺Foxp3⁺ (T_{H}2-like) T regulatory cell, a IL-17⁺Foxp3⁺ (T_{H}17-like) T regulatory cell, a T_{H}2 cell, and a T_{H}17 cell.
19) The method of any of the preceding paragraphs, wherein circulation is decreased by at least 50%, at least 60%, at least 70%, at least 80%, at least 90%, or more as compared to an appropriate control.
20) The method of any of the preceding paragraphs, wherein the agent is administered at a concentration of 8 mg/kg or 10 mg/kg.
21) The method of any of the preceding paragraphs, further comprising administering at least a second asthma therapeutic.
22) A method of treating asthma, the method comprising administering to a subject in need thereof an effective amount of tocilizumab.
23) The method of any of the preceding paragraphs, wherein the asthma is pediatric asthma.
24) The method of any of the preceding paragraphs, wherein the asthma is non-atopic asthma.
25) The method of any of the preceding paragraphs, wherein the asthma is severe, persistent asthma.
26) The method of any of the preceding paragraphs, further comprising, prior to administration, diagnosing a subject with having asthma.
27) The method of any of the preceding paragraphs, wherein the subject has a mutation in the IL4R gene.
28) The method of any of the preceding paragraphs, wherein the subject is a homozygous for TL4R dominant allele.
29) The method of any of the preceding paragraphs, wherein the subject is a homozygous for IL4R mutant allele.
30) The method of any of the preceding paragraphs, further comprising, prior to administration, identifying a subject as having a mutation in the IL4R gene.
31) The method of any of the preceding paragraphs, further comprising, prior to administration, receiving results that identify a subject as having a mutation in the IL4R gene.
32) The method of any of the preceding paragraphs, wherein tocilizumab is administered at a concentration of 8 mg/kg or 10 mg/kg.
33) The method of any of the preceding paragraphs, wherein tocilizumab is administered once every 2 weeks or once every 4 weeks.
34) The method of any of the preceding paragraphs, wherein administration decreases circulation of a cell selected from the group consisting of: a IL-4⁺Foxp3⁺ (T_{H}2-like) T regulatory cell, a IL-17⁺Foxp3⁺ (T_{H}17-like) T regulatory cell, a T_{H}2 cell, and a T_{H}17 cell.
35) The method of any of the preceding paragraphs, wherein circulation is decreased by at least 50%, at least 60%, at least 70%, at least 80%, at least 90%, or more as compared to an appropriate control.
36) The method of any of the preceding paragraphs, further comprising administering at least a second asthma therapeutic.
37) A composition comprising an agent that inhibits IL-6 signaling.
38) The composition of any of the preceding paragraphs, further comprising a pharmaceutically acceptable carrier.

### EXAMPLES

### Example 1

Asthma is a chronic lung inflammatory disease with multiple phenotypic manifestations, underlined by several disease endotypes that reflect distinct pathophysiological mechanisms¹. A T helper cell type 2 high (T_{H}2^{High}) and mixed T_{H}2/T_{H}17 endotypes have been associated with severe asthma². The former is characterized by an eosinophilic and the latter by a mixed eosinophilic and neutrophilic airway inflammation, with the T_{H}2/T_{H}17 endotype manifesting as a difficult-to-control, steroid-resistant disease^{3,4}. Past research has demonstrated high sputum levels of IL-6 in patients with mixed eosinophilic/neutrophilic airway inflammation⁵. IL-6 blockade has been proposed as a treatment for asthma⁶. The IL-4 receptor alpha chain variant R576 (IL-4Ra-R576) drives mixed T_{H}2/T_{H}17 airway inflammation^{7, 8}. Treatment of Il4ra^{R576} mice with an anti-IL-6 monoclonal antibody (mAb) protected against severe airway inflammation.⁵ Furthermore, there has been no reported cases of IL-6 pathway blockade for pediatric asthma. Described herein is the response of two patients with severe persistent, non-atopic asthma with evidence of T_{H}2/T_{H}17 inflammation treated with tocilizumab, a humanized anti-IL-6 receptor (IL-6R) mAb.

Patient 1: This is a 6-year-old boy with severe persistent, non-atopic brittle asthma homozygous for the IL4R^{R576} allele (mutant allele). He had severe life-threatening asthma with 18 intensive care unit (ICU) admissions, four requiring intubations (of which two also required isoflurane), and multiple other ICU admissions requiring non-invasive positive pressure ventilation. Past workup demonstrated negative testing to aeroallergens (skin prick testing [SPT] and serum allergen-specific IgE [sIgE]), normal total serum IgE, normal immune evaluation, negative sweat test result for cystic fibrosis and mild peripheral eosinophilia despite oral steroids (peak AEC 1030 cells/μL). Modified barium swallow (MBS) showed deep laryngeal penetration of thin liquids and rigid bronchoscopy revealed type 1 laryngeal cleft. Flexible bronchoscopy and bronchoalveolar lavage (BAL) revealed columnar epithelium admixed with numerous eosinophils and scattered neutrophils, macrophages, and lymphocytes. Testing for anti-neutrophil cytoplasmic antibodies was negative, and he did not fulfill criteria for Churg-Strauss syndrome. Despite taking fluticasone-salmeterol, montelukast, azithromycin, prednisolone (7.5 mg QOD), omeprazole, and nectar-thickened feeds, he developed on November 2015 severe status asthmaticus requiring prolonged ICU admission. During this admission he underwent laryngeal cleft repair, following which his repeat MBS was normal and thickened feeds were successfully discontinued. Theophylline and intravenous immunoglobulins [IVIG, given at 1 g/kg every four weeks (q4wk)] were added to his treatment regimen. However, his medical insurance denied coverage of Omalizumab, Mepolizumab and Dupilumab therapies. While he appeared to improve following the addition of theophylline and IVIG, he had another severe asthma exacerbation in September 2016. Given his ongoing admissions and known T_{H}2/T_{H}17 asthma endotype, he started tocilizumab on 10/27/16 at 10 mg/kg IV q4wk. Six months later, the patient was admitted to ICU in status asthmaticus, so tocilizumab was increased to 8 mg/kg/dose q2wk and IVIG to 1 gram/kg q2wk. On this regimen he developed neutropenia (ANC 680 cells/L). Accordingly, the tocilizumab dose was readjusted to 10 mg/kg q4wk, on which he is currently maintained, in addition to budesonide/formoterol (160 mcg-4.5 mcg, 2 puff twice daily), montelukast (10 mg daily), azithromycin (200 mg three times/wk), theophylline (450 mg daily), prednisolone (9 mg every other day), and IVIG 1 gm/kg q2wk.

Overall, the patient exhibited sustained clinical and immunologic responses to tocilizumab over the past 19 months. Clinically, he had decreased hospital admissions and inpatient hospital days, increased asthma control test (ACT) scores and improved pulmonary function testing (FEV1 increased from 76% to 99% of predicted) (Table 1). He has also had a marked immunological response to tocilizumab with decreased circulating IL-4⁺Foxp3⁺ (T_{H}2-like) and IL-17⁺Foxp3⁺ (T_{H}17-like) Treg cells, implicated in disease pathogenesis (FIGS. 26A and 26C) 9 as well as decreased T_{H}2 and T_{H}17 effector cells (FIG. 27A).

Patient 2: This is a 5-year-old boy with mild atopic dermatitis, eosinophilic esophagitis, and severe persistent, non-atopic asthma who was homozygous for the dominant IL4R^{Q576} allele. He had persistent severe symptoms despite taking mometasone/formoterol, fluticasone, montelukast, theophylline, prednisolone (5 mg every other day), and omeprazole. Azithromycin was discontinued due to lack of clinical benefit. Theophylline was discontinued due to side effects. Evaluation revealed negative SPT and sIgE to aeroallergens, normal total IgE, reassuring immune evaluation, sweat test not suggestive of cystic fibrosis, normal ciliary biopsy, and negative ABPA work up. He had mild peripheral eosinophilia despite oral steroids (peak AEC: 1440 cells/μL), without evidence for Churg-Strauss syndrome. MBS showed deep laryngeal penetration of thin liquids and rigid bronchoscopy revealed type 1 laryngeal cleft. He underwent flexible bronchoscopy and BAL that demonstrated airway eosinophilia. BAL culture grew moderate Streptococcus pneumoniae, for which he was treated with Augmentin. Nevertheless, he continued to have persistent exacerbations requiring oral steroids. He was not a candidate for omalizumab or IVIG therapy. He started tocilizumab on 2/23/17 at 10 mg/kg IV q4wk. Due to ongoing asthma symptoms, tocilizumab was increased to 8 mg/kg q2wk on May 2017. He had one episode of neutropenia (ANC: 840 cells/L) that spontaneously resolved after tocilizumab was held for 2 wk. He discontinued tocilizumab on 8/28/17 per family request.

While on tocilizumab, the patient demonstrated favorable clinical response with decreased hospital admissions (Table 1), and was weaned off oral prednisolone. Flow cytometric analysis at baseline demonstrated T_{H}2^{high} skewing affecting his Treg and Teff cells with a lesser T_{H}17 cell response as compared to patient 1 (FIG. 26B). Tocilizumab therapy suppressed his circulating IL-4⁺Foxp3⁺ (T_{H}2-like) and IL-17⁺Foxp3⁺ (T_{H}17-like) Treg cells (FIGS. 26B and 26D) and T_{H}2 and T_{H}17 cells (FIG. 27B). His immunological improvement remained sustained at 4 months post tocilizumab therapy, the last time his studies were repeated.

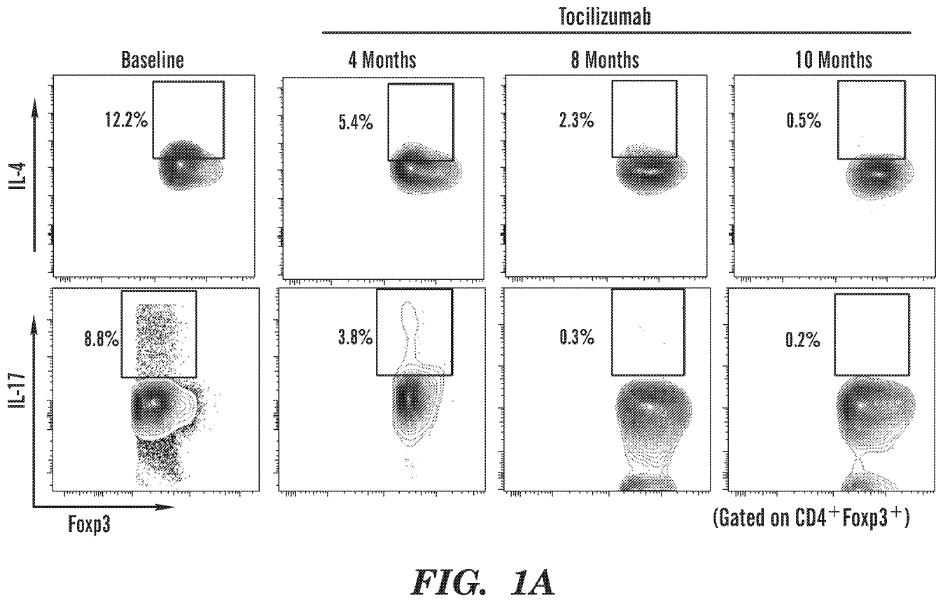

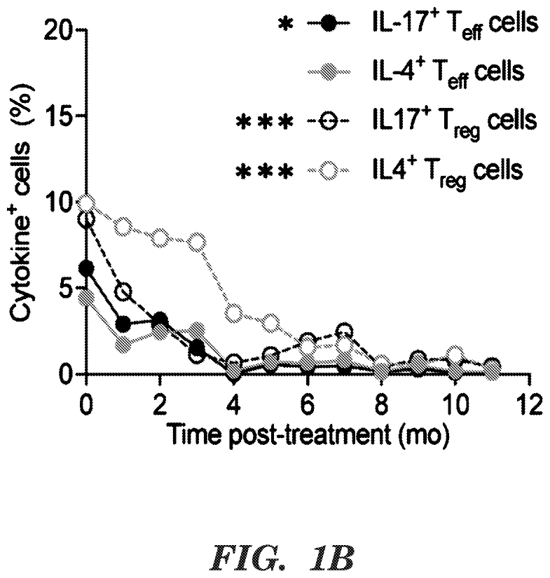

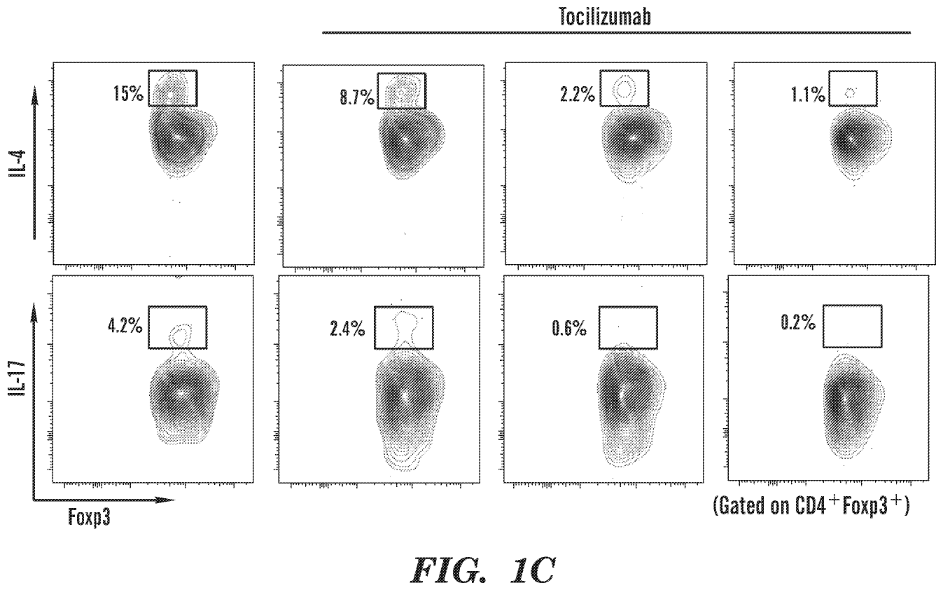

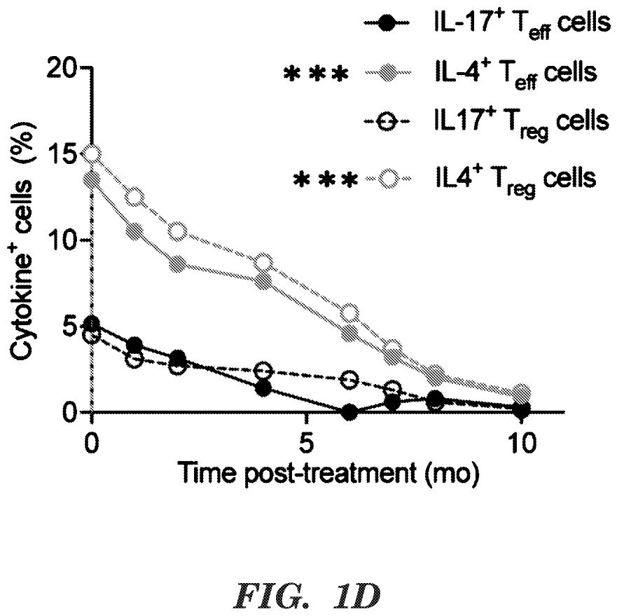

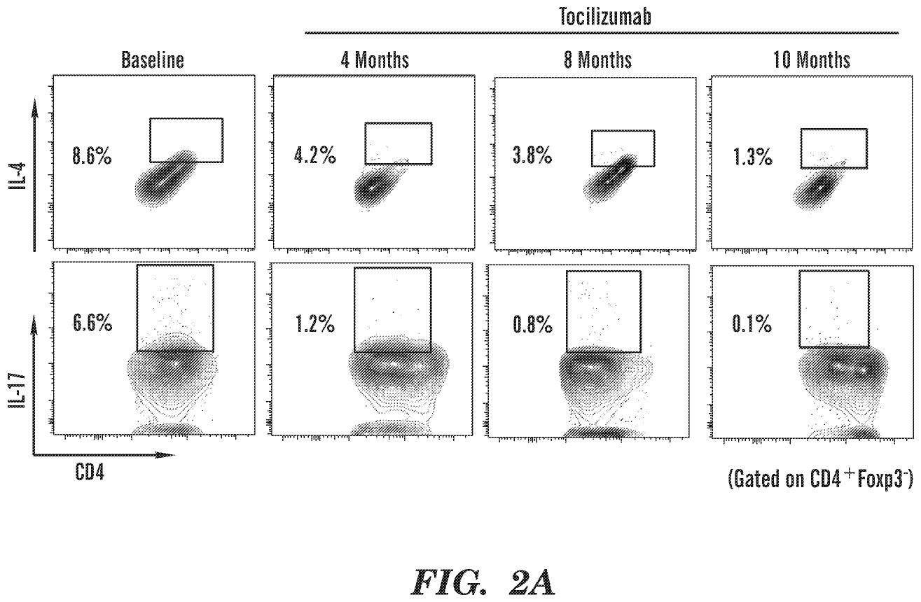

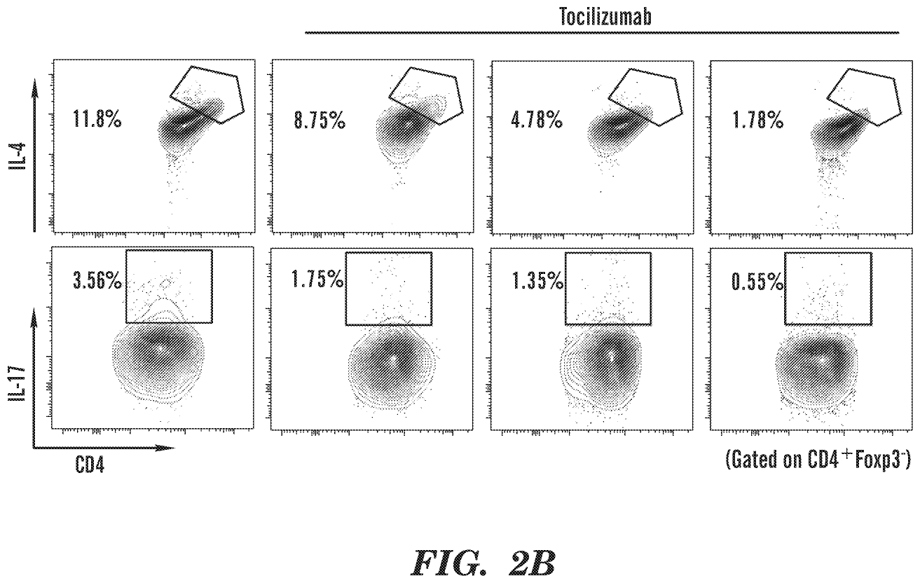

Tocilizumab therapy suppresses Th2 and Th17 cytokine expression in T effector (Teff) and T regulatory cells of severe asthmatics. Flow cytometric analysis of peripheral blood lymphocytes demonstrated that at baseline, just prior to the start of Tocilizumab therapy, patient 1 had appreciable frequencies of circulating IL-4⁺CD4⁺Foxp3⁺ (T_{H}2-like) and IL-17⁺CD4⁺Foxp3⁺ (T_{H}17-like) regulatory T (Treg) cells, implicated in disease pathogenesis (FIGS. 1A and 1B), as well as CD4⁺Foxp3-T_{H}2 and T_{H}17 T effector (Teff) cells (FIG. 1B), albeit with more bias towards Th17 cell response (IL-17). Tocilizumab therapy suppressed his circulating IL-4⁺CD4⁺Foxp3⁺ (T_{H}2-like) and IL-17⁺CD4⁺Foxp3⁺ (T_{H}17-like) Treg cells and T_{H}2 and T_{H}17 Teff cells (FIGS. 1A and 1B). His immunological responses remained sustained at 12 months post the start of tocilizumab therapy.

Patient 2 also demonstrated circulating IL-4⁺CD4⁺Foxp3⁺ (T_{H}2-like) and IL-17⁺CD4⁺Foxp3⁺ (T_{H}17-like) Treg and T_{H}2 and T_{H}17 T effector cells, but with more bias towards Th2 cytokine expression (IL-4⁺). He had a marked immunological response to tocilizumab with decreased circulating IL-4⁺CD4⁺Foxp3⁺ (T_{H}2-like) and IL-17⁺CD4⁺Foxp3⁺ (T_{H}17-like) Treg cells, implicated in disease pathogenesis (FIGS. 1C and 1D), as well as decreased T_{H}2 and T_{H}17 effector cells (FIG. 1D). His immunological responses remained sustained at 10 months post the start of tocilizumab therapy. Overall, Tocilizumab therapy was effective in both patients, in association with suppression of T_{H}2/T_{H}17 cytokine expression in Treg and Teff cells.

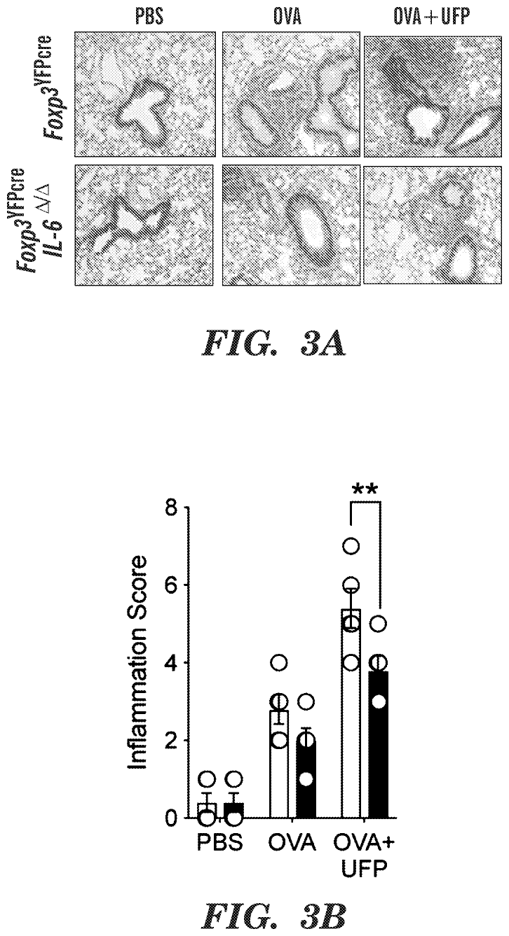

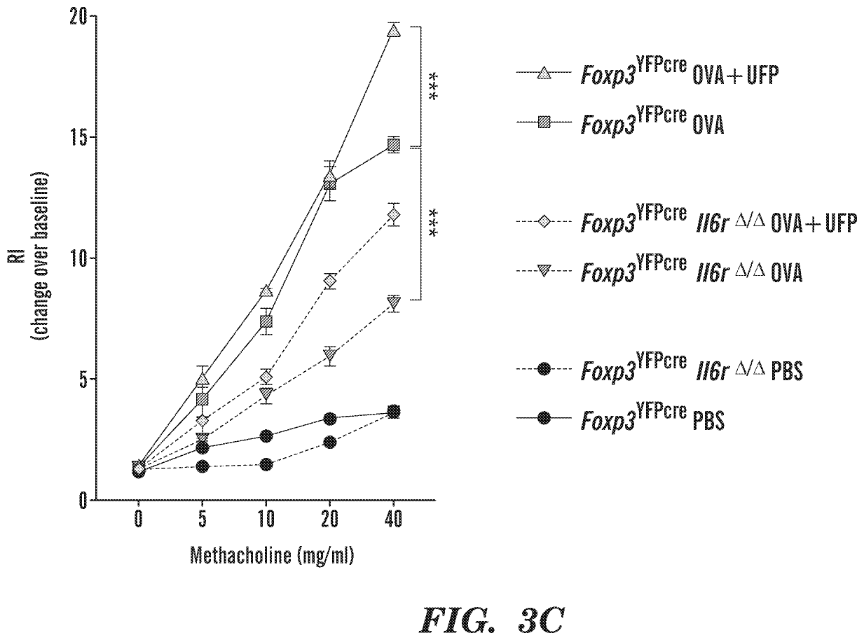

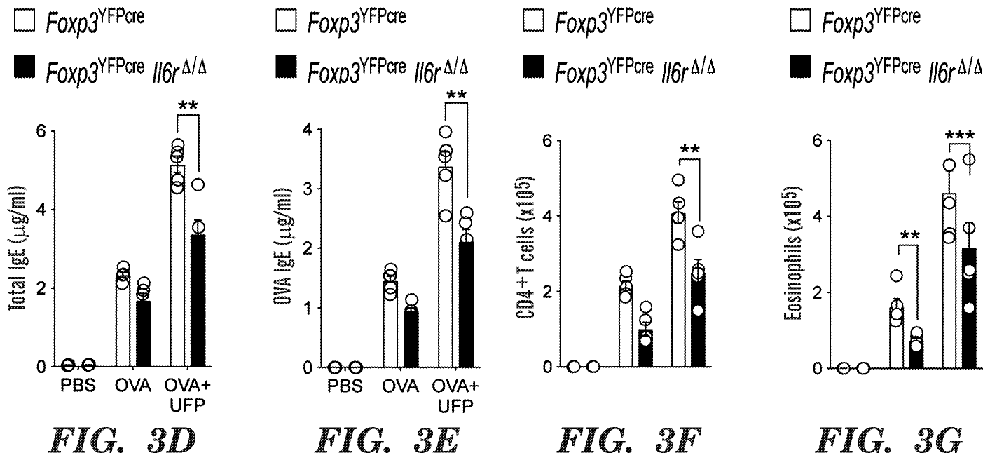

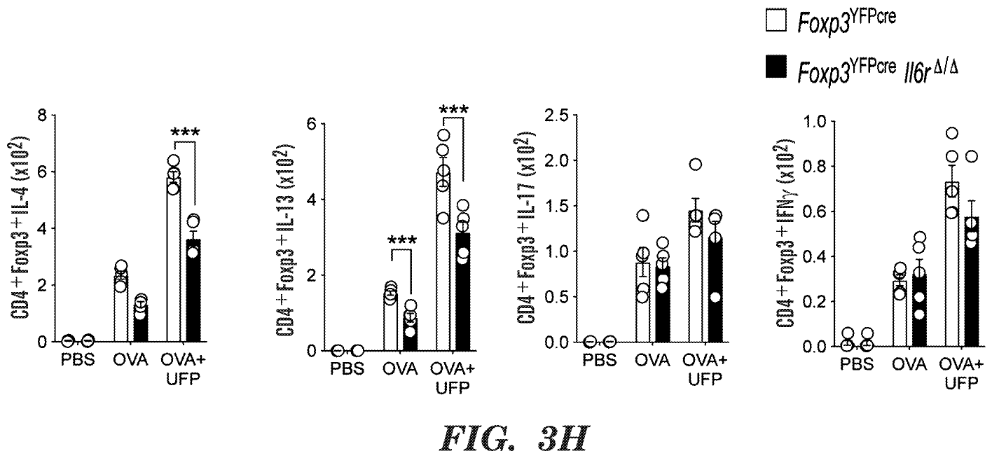

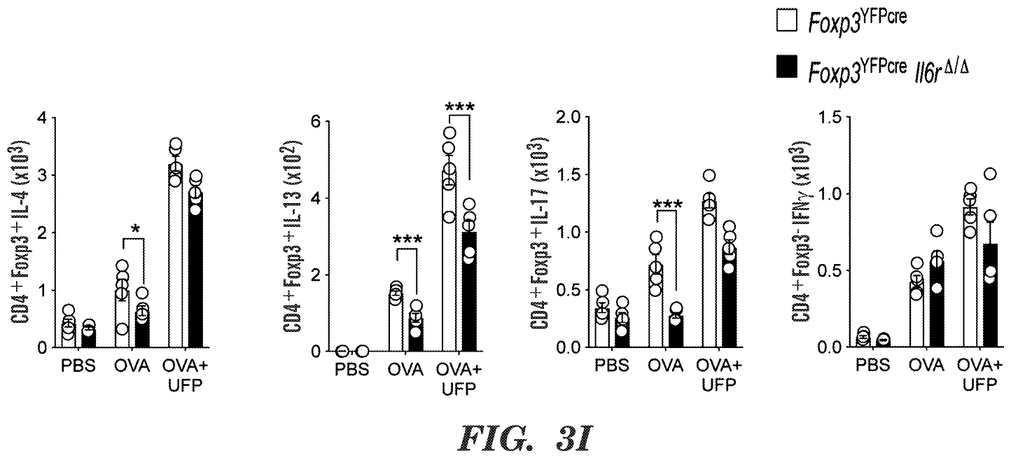

Treg cell-specific deletion of Il6ra attenuates allergic airway inflammation in mice and reduces Notch4 expression on lung Treg cells. The IL-6 receptor is composed of a ligand binding chain (IL-6Rα chain) and a signal transducing chain (IL-6ST or gp130). To examine the relationship between IL-6R signaling in Treg cells and Notch4 expression in the context of allergic airway inflammation, we employed mice with a floxed Il6ra allele that specifically deleted in Treg cells using a Foxp3-driven Cre recombinase (Foxp3^{YFPCre}). OVA-sensitized mice with deleted Il6ra in their Treg cells (Foxp3^{YFPCre}Il6ra^{Δ/Δ}) exhibited a markedly attenuated airway inflammatory response when sensitized with OVA and then challenged with either OVA or OVA/UFP, with decreased airway resistance, tissue inflammation, eosinophilia and OVA-specific IgE responses as compared to mice with Il6ra-sufficient Treg cells (FIG. 3A-3G). The attenuation in the airway inflammatory response in Foxp3^{YFPCre}Il6ra^{Δ/Δ} mice was somewhat less marked than that observed with Foxp3^{YFPCre}Notch4^{Δ/Δ} mice, with the airway inflammatory response remaining somewhat elevated in the OVA+UFP-treated Foxp3^{YFPCre}Il6ra^{Δ/Δ} mice (FIG. 3H-3I).

### CONCLUSION

In summary, both patients demonstrated clinical and immunological responses to tocilizumab therapy with no adverse infections despite developing mild neutropenia that spontaneously resolved. Neither patients' peripheral eosinophilia was impacted by tocilizumab therapy. Patient 1, homozygous for the IL4R^{R576} allele, continues on tocilizumab with excellent clinical and immunologic response. Patient 2, homozygous for the dominant IL4R^{Q576} allele, discontinued tocilizumab but also demonstrated clinical and immunological improvement

Tocilizumab therapy is effective in patients with severe persistent, steroid-resistant asthma by virtue of suppressing both T_{H}2 and T_{H}17 cell responses. Results presented herein also demonstrate the utility of monitoring circulating T_{H}2 and T_{H}17 cells in asthmatic patients in assessing therapeutic responses.

TABLE 1
Clinical response: Asthma control test, pulmonary function testing, and hospital admissions.

| | Table 1 - Patient 1 | | | | | | | | |
|---|---|---|---|---|---|---|---|---|---|
| | | | | | Admissions | | | | |
| | Pulmonary Function Testing | | | | | General | | | Inpatient |
| Date | ACT | FVC | FEV1 | FEV1/FVC | Year | Inpatient | ICU | Total | Days |
| Jul. 22, 2016†: | | | | | | | | | |
| pre | 21 | 1.10 (87%) | 0.82 (71%) | 74 (81%) | 2014 | 0 | 5 | 5 | 12 |
| post | | 1.46 (115%) | 1.10 (95%) | 75 (82%) | | | | | |
| Oct. 27, 2016* | 27 | 1.48 (114%) | 0.9 (76%) | 61 (67%) | 2015 | 0 | 5 | 5 | 68 |
| Dec. 28, 2016 | 27 | 0.98 (75%) | 0.58 (49%) | 59 (69%) | 2016** | 0 | 2 | 2 | 14 |
| May 17, 2017 | 27 | 1.68 (89%) | 1.28 (81%) | 76 (86%) | 2016*** | 0 | 0 | 0 | 0 |
| May 26, 2017 | 27 | 1.77 (125%) | 1.16 (91%) | 66 (73%) | 2017 | 1 | 1 | 2 | 17 |
| Dec. 13, 2017 | ACT | 1.90 (125%) | 1.30 (99%) | 68 (77%) | 2018_{(1/18-11/18)} | 0 | 0 | 0 | 0 |

Table 1 - Patient 2

| | | | | | Admissions | | | |
|---|---|---|---|---|---|---|---|---|
| | | | | | | General | | |
| Date | ACT | FVC | FEV1 | FEV1/FVC | Year | Inpatient | ICU | Total |
| Feb. 23, 2017* | 13** | 1.27 (118%) | 1.22 (122%) | 96 (103%) | | | | |
| Jul. 25, 2017 | 20 | 1.51 (126%) | 1.16 (106%) | 77 (84%) | 2015 | 1 | 0 | 1 |
| Aug. 10, 2017 | 20 | 1.53 (128%) | 1.22 (111%) | 79 (86%) | 2016 | 4 | 3 | 7 |
| Aug. 28, 2017 | 22 | 1.62 (136%) | 1.33 (121%) | 82 (89%) | 2017 | 1 | 0 | 1 |
| May 17, 2018 | 21 | 1.66 (123%) | 1.32 (109%) | 80 (88%) | 2018_{(1/18-6/18)} | 0 | 0 | 0 |

For Table 1, Patient 1;
†Response to bronchodilator therapy. Pre and Post: pulmonary function tests prior to and post therapy,
*Initiation of tocilizumab. Rows “Oct. 27, 2016”, “2014”, “2015”, and “2016” indicate time period prior to initiation of tocilizumab.
For Table 1, Patient 2;
*Initiation of tocilizumab;
**Obtained at clinic appointment on Oct. 21, 2016. Rows “Feb. 23, 2017”, “2015”, and “2016” indicate time period prior to initiation of tocilizumab. For ACT scores for children under 12, the maximal ACT score is 27 (Nathan R A et al. J Allergy Clin Immunol. 2004; 113: 59-65).

### Methods and Materials

Study approval. Studies on patient peripheral blood samples were all performed at the Boston Children's Hospital and were approved by the Institutional Review Board.

Antibodies. Flow cytometry and intracellular staining. Single-cell suspensions were stained with the indicated antibodies (Ab) and analyzed on LSRIIFortessa cytometer (Becton Dickinson). Cytokine expression in CD4⁺ T cells was determined by stimulating cells with PMA (20 ng/ml) plus ionomycin (1 μg/ml) for 4 hours in the presence of Golgi-plug (BD Biosciences) followed by intracellular staining for the respective cytokine using the eBioscience Fixation/Permbealization buffer following the manufacturer's instructions. Fluorescence-conjugated mAbs used were obtained from BD Biosciences, Biolegend and eBioscience. Anti-CD3-APC-Cy7, (H1T3a), anti-CD4-PerCP-Cy5.5 and PE (PRA-T4), anti-CD25-PE (CD25-4E3), anti-CD127-PE-Cy7 (A019D5), anti-CRTH2-FITC (BM16), anti-CXCR3-APC (G025H7), anti-CCR4-BV605 (L29H14), anti-CCR6-Amcyan (G034E3). For intracellular staining, the following mAbs were used from BD Biosciences, Biolegend and eBioscience, anti-IFNG-PE-Cy7 (45.B3) anti-IL-13-PerCP-Cy5.5 (JE510-SA2), Anti-IL-4-BV605 (MP4-25D2), anti-IL-17-APC (BL 168), anti-FOXP3-Pacific Blue (PCH101).

Cell preparation. Blood was obtained from the patients after a written consent. PBMCs were isolated using Ficoll (GE-Healthsciences). Shortly, 4 mL of Ficoll were layered in a 15 mL tube. Afterwards, the blood will be layered on top of ficoll very slowly to build two separate phases. The ficoll/blood mixture were centrifuged on 300 g for 20 min without breaks to have the PBMCs caught in the middle layer. The cells were aspirated into a new 15 mL tube and washed twice with 10 mL PBS. The cell pellet was then used for the PMA/Ionomycin/Golgi-plug stimulation and FACs staining.

Data analysis. The flow cytometric analysis of the data was done using Flowjo software (FlowJo, LLC). The graphs and statistical analysis were done using GraphPad Prism version 7.00 for Windows, GraphPad Software, La Jolla Calif. USA (available on the world wide web at www.graphpad.com). The time course of cytokine expression post-therapy was analyzed by two-way ANOVA with Tukey post-test analysis. Within each cell type and for each cytokine, the values obtained post therapy were compared to the pre-therapy baseline value. A p value <0.05 was considered statistically significant.

### REFERENCES—EXAMPLE 1

1. Lotvall J, Akdis C A, Bacharier L B, Bjermer L, Casale T B, Custovic A, et al. Asthma endotypes: a new approach to classification of disease entities within the asthma syndrome. J Allergy Clin Immunol 2011; 127:355-60.
2. Agache I, Akdis C A. Endotypes of allergic diseases and asthma: An important step in building blocks for the future of precision medicine. Allergol Int 2016; 65:243-52.
3. Irvin C, Zafar I, Good J, Rollins D, Christianson C, Gorska M M, et al. Increased frequency of dual-positive TH2/TH17 cells in bronchoalveolar lavage fluid characterizes a population of patients with severe asthma. J Allergy Clin Immunol 2014; 134:1175-86 e7.
4.Bhakta N R, Erle D J. IL-17 and “TH2-high” asthma: Adding fuel to the fire? J Allergy Clin Immunol 2014; 134:1187-8.
5. Chu D K, Al-Garawi A, Llop-Guevara A, Pillai R A, Radford K, Shen P, et al. Therapeutic potential of anti-IL-6 therapies for granulocytic airway inflammation in asthma. Allergy Asthma Clin Immunol 2015; 11:14.
6. Rincon M, Irvin C G. Role of IL-6 in asthma and other inflammatory pulmonary diseases. Int J Biol Sci 2012; 8:1281-90.
7. Massoud A H, Charbonnier L M, Lopez D, Pellegrini M, Phipatanakul W, Chatila T A. An asthma-associated IL4R variant exacerbates airway inflammation by promoting conversion of regulatory T cells to TH17-like cells. Nat Med 2016; 22:1013-22.
8. Abdel-Gadir A, Massoud A H, Chatila T A. Antigen-specific Treg cells in immunological tolerance: implications for allergic diseases. F1000Res 2018; 7:38.
9. Noval Rivas M, Chatila T A. Regulatory T cells in allergic diseases. J Allergy Clin Immunol 2016; 138:639-52.

### Example 2

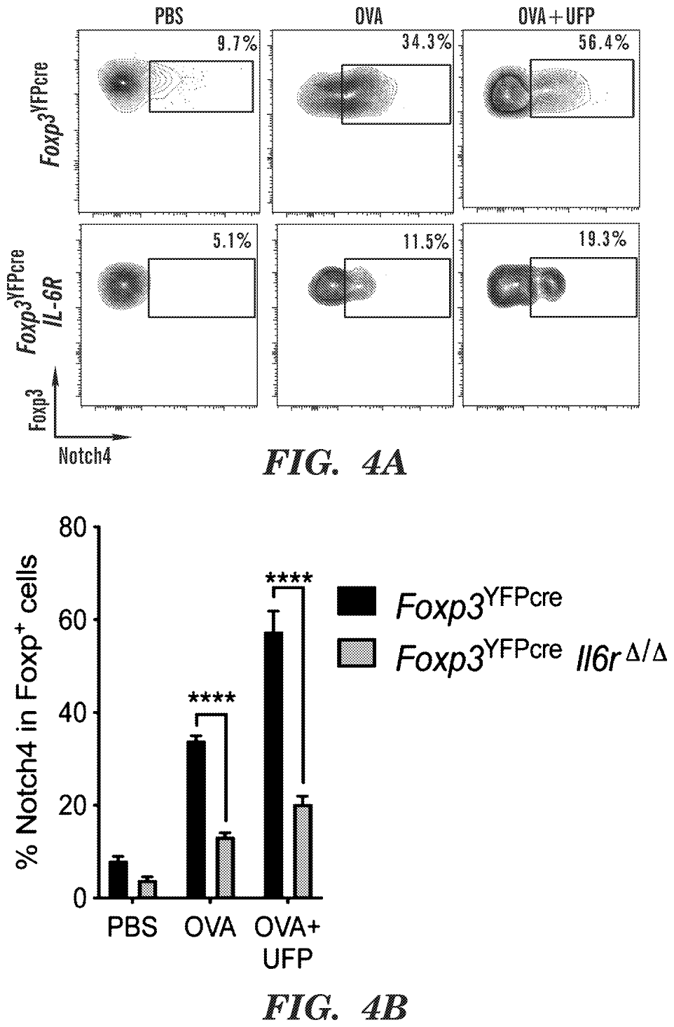

Treg cell-specific deletion of Il6ra attenuates allergic airway inflammation in mice and reduces Notch4 expression on lung Treg cells. Our previous studies in mice have shown that blockading the IL-6/IL-6 receptor (IL-6R) interaction with an anti-IL-6 monoclonal antibody (mAb) protected mice expressing the IL-4R-R576a chain variant (Il4raR576) against exacerbated allergic airway inflammation induced by allergens. The IL-6 receptor is composed of a ligand binding IL-6Rα chain, encoded by the Il6ra gene, and a signal transducing chain (IL-6ST or gp130). To examine the contribution of IL-6R signaling blockade in CD4+Foxp3+T regulatory (Treg) cells in the attenuation of allergic airway inflammation induced by both allergens and traffic-related ultra fine particle pollutants (UFP), we employed mice with a floxed Il6ra allele that was specifically deleted in Treg cells using a Foxp3-driven Cre recombinase (Foxp3YFPCre). Foxp3YFPCreIl6raΔ/Δ mice (with Treg cell-specific deletion of Il6ra) and control Foxp3YFPCre (whose Treg cells were Il6ra-sufficient) were either sham sensitized with phosphate buffered saline (PBS) or sensitized with the allergen chicken egg ovalbumin (OVA). They were then challenged with OVA or with OVA together with UFP (OVA+UFP), and the respective mouse groups were analyzed for their airway inflammatory responses. Compared to similarly treated control Foxp3YFPCre mice, the OVA-sensitized and OVA or OVA+UFP-challenged Foxp3YFPCreIl6raΔ/Δ mice exhibited markedly attenuated airway inflammatory responses. The Foxp3YFPCreIl6raΔ/Δ mice had decreased lung tissue inflammation, airway resistance, total and OVA-specific IgE responses, and lung tissue CD4+ T cells and eosinophil infiltration (FIG. 3A-3G). Furthermore, the Treg cells of allergen or allergen pollutant treated Foxp3YFPCreIl6raΔ/Δ mice expressed decreased amounts of pro-allergic inflammatory cytokines IL-4, IL-13 and IL-17 as compared to those control mice, indicative of their protection from degeneracy into pathogenic T effector-like cells by the Il6ra deletion. The more effective Treg cell function in the Foxp3YFPCreIl6raΔ/Δ mice was also reflected by decreased numbers of activated CD4+Foxp3-T effector cells in the lung tissue and their decreased expression of the pro-allergic inflammatory cytokines (FIGS. 4A and 4B).

Overall, these results indicated that a major mechanism of action of IL-6Ra, chain blockade by Tocilizumab involved the augmentation of immune tolerance in the airways by enabling Treg cell function and preventing their degeneracy into Teff cell-like phenotype.

### Example 3

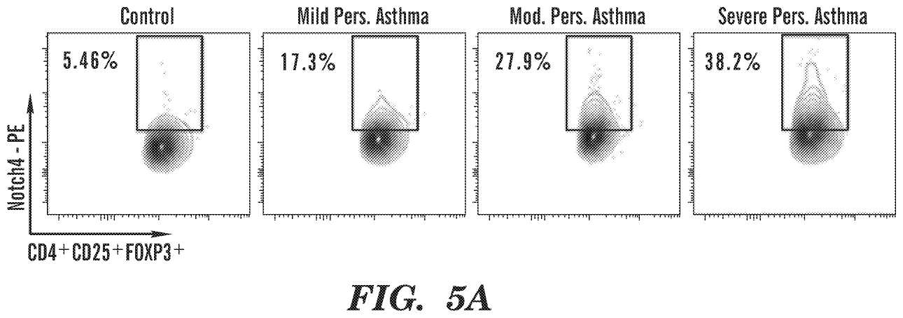

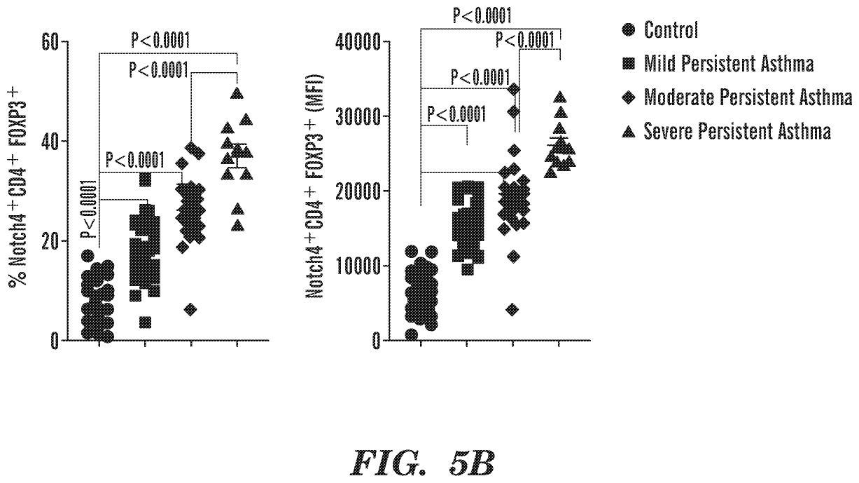

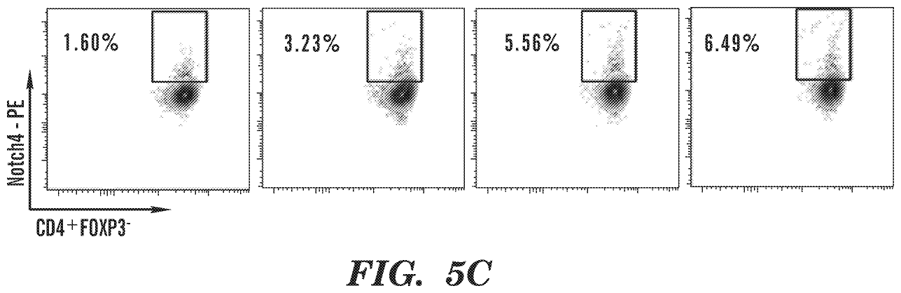

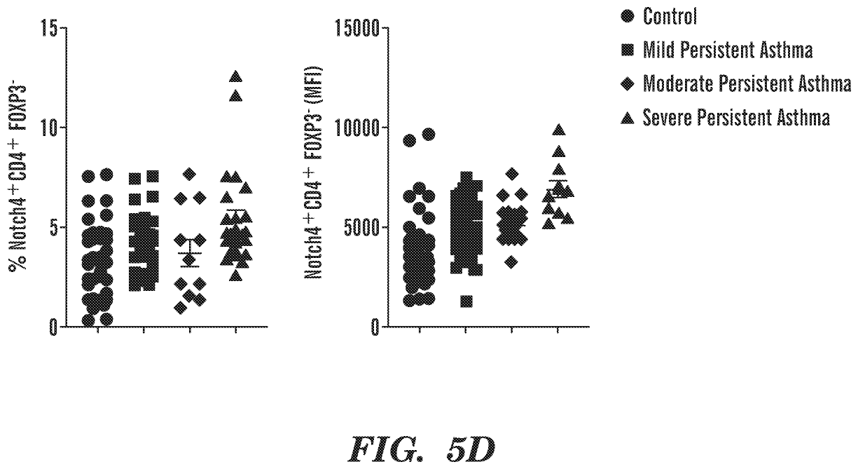

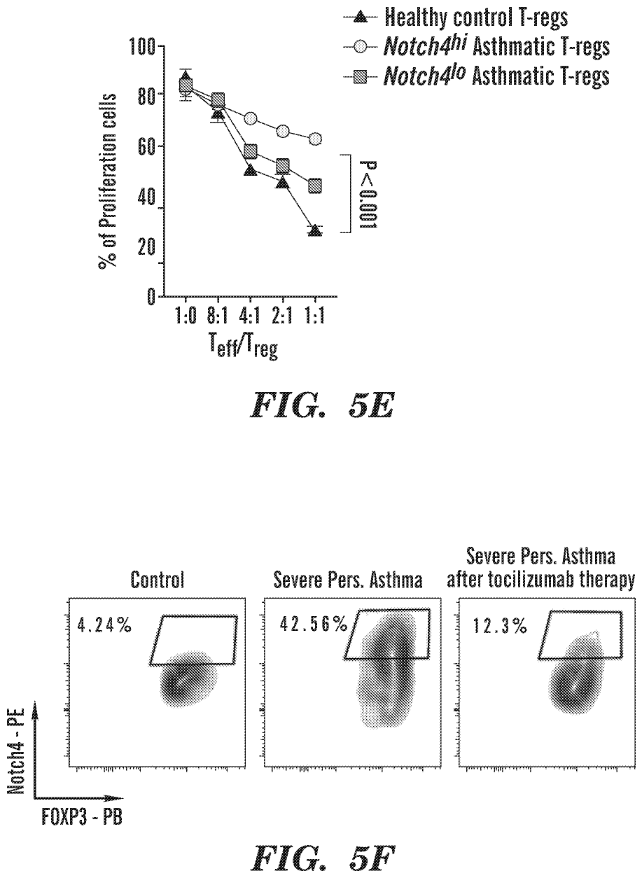

IL-6 promotes the induction of Notch4 on Treg cells in asthma. To determine the role of Notch receptors in asthma, we analyzed the expression of Notch1-4 on peripheral blood mononuclear cells (PBMC) of pediatric age asthmatic and control subjects (age 2-18 years). Asthma severity was defined based on Asthma severity is graded based on recommendations from the National Asthma Education and Prevention Program, Third Expert Panel on the Diagnosis and Management of Asthma¹. Results revealed that asthmatics had elevated frequencies of circulating Notch4⁺ Treg cells (FIG. 5A). Both the cell frequencies and expression intensity progressively increased as a function of asthma severity, reaching up to 50% of circulating Treg cells in severe asthmatics (Figure. 5B). In contrast, Notch4 expression on circulating CD4⁺ T_{conv} was low and remained relatively flat as a function of asthma severity (Figure. 5C, 5D). The contribution of Notch4 signaling to Treg cell dysfunction was ascertained by the demonstration that Notch4^{high} peripheral blood Treg cells poorly suppressed in vitro T cell proliferation as compared to Notch4^{low}Treg cells isolated from the same asthmatic subjects or to Treg cells isolated from healthy control subjects, which were overwhelmingly Notch4^{low} (Figure. 5E). Consistent with the results shown in FIG. 5A, the patient manifested very high expression of Notch4 on his circulating Treg cells (FIG. 5F). Treatment with Tocilizumab at 8-10 mg/kg/week for 3 months was associated with a dramatic reduction in the expression of Notch4 on the patient Treg cells (FIG. 5F).

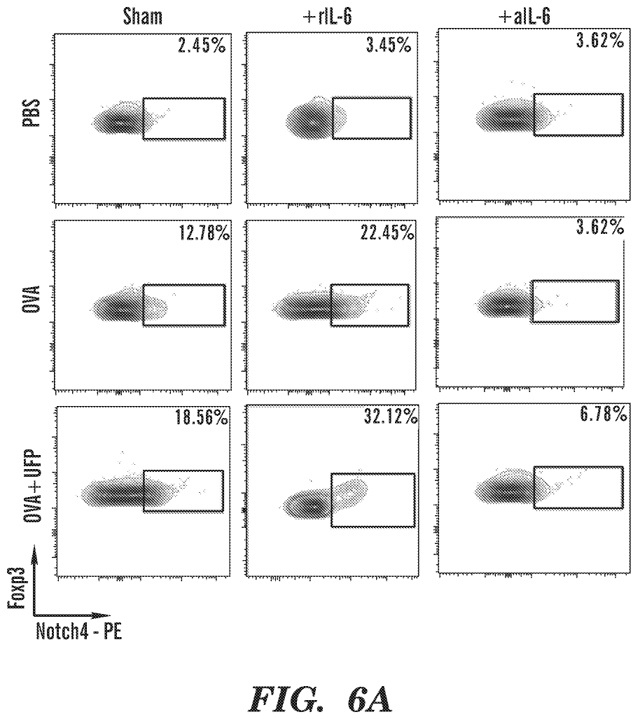

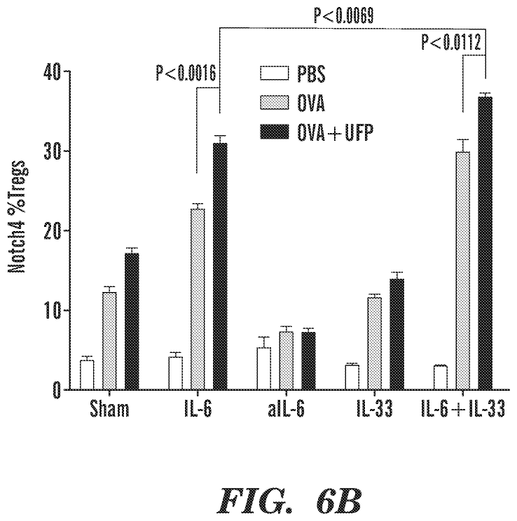

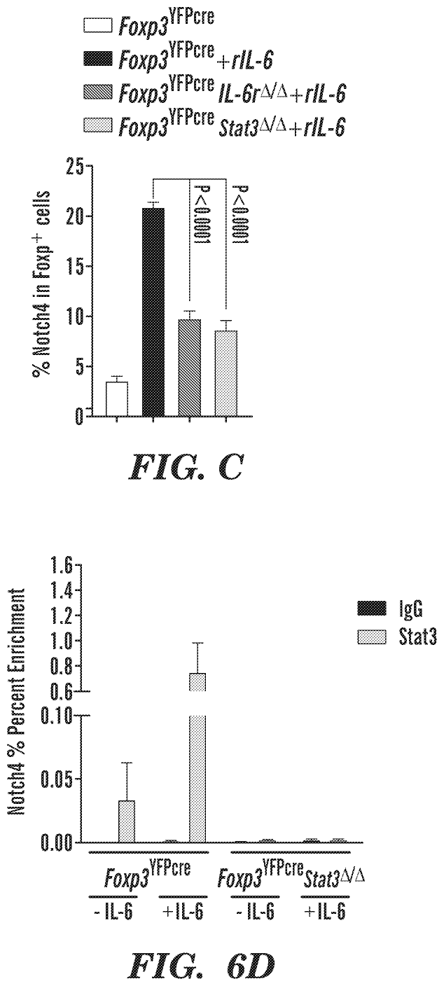

To determine the mechanisms involved in the induction of Notch4 on Treg cells, we employed an in vitro Treg cell differentiation assay involving naïve mouse transgenic T cells expressing the OVA peptide 323-339-specific T cell receptor OT-II. The cells were incubated with alveolar macrophages isolated from mouse lung tissue that were either sham pulsed or pulsed with the OVA peptide, alone or together with UFP. Expression of Notch4 on differentiated Treg cells was monitored flow cytometry. Treg cell differentiation was induced upon the co-culture of OT-II T cells with OVA or OVA+UFP pulsed but not sham pulsed macrophages. Notch4 was found specifically expressed on differentiated Treg cells induced by activation with OVA or OVA+UFP (FIG. 6A), whereas naïve T cells that failed to differentiate into Treg cells expressed very little Notch4. We have previously shown that UFP induces IL-6 production in alveolar macrophages 2 Importantly, addition of TL-6 to the co-culture markedly upregulated the expression of Notch4 on Treg cells (FIG. 6B), whereas the addition of an anti-IL-6 mAb to the co-cultures suppressed the expression of Notch4 (FIG. 6B). Another cytokine implicated in airway inflammation, IL-33, did not induce Notch4 but further upregulated the induction of Notch4 by IL-6 (FIG. 6B). These results indicated that Notch4 expression on induced Treg cells in the lung is enabled by IL-6 in synergy with IL-33. Furthermore, we have put naïve T-cells under Treg differentiation conditions, once the cells were differentiated, we have added rIL-6 to these cells. In WT conditions, Notch4 expression was upregulated, while in IL-6r deficient or Stat3 deficient Treg cells, Notch4 expression was suppressed (FIG. 6C). This shows that Notch4 expression is dependent on Il-6/Stat3 to be induced. Moreover, we have measured percentage enrichment of Notch4 promoter using stat3 monoclonal chromatin immunoprecipitation (ChIP) antibody. IL-6 stimulation of Treg cells induced chromatin modification at the Notch4 promoter site, rendering the promoter open and ready for transcription, due to the binding of Stat3 at the promoter site (FIG. 6D). Overall, we are able to show that Notch4 induction is completely dependent on IL-6r on Treg cells.

1) A method of treating asthma, the method comprising administering to a subject in need thereof an effective amount of an agent that inhibits the IL-6 signaling.

2) The method of claim 1, wherein the asthma is pediatric asthma.

3) The method of claim 1 or 2, wherein the asthma is non-atopic asthma.

4) The method of any of claims 1-3, wherein the asthma is severe, persistent asthma.

5) The method of claim 1, further comprising, prior to administration, diagnosing a subject with having asthma.

6) The method of any of claims 1-5, wherein the subject has a mutation in the IL4R gene.

7) The method of claim 6, wherein the subject is a homozygous for IL4R dominant allele.

8) The method of claim 6, wherein the subject is a homozygous for IL4R mutant allele.

9) The method of claim 1, further comprising, prior to administration, identifying a subject as having a mutation in the IL4R gene.

10) The method of claim 1, further comprising, prior to administration, receiving results that identify a subject as having a mutation in the IL4R gene.

11) The method of claim 1, wherein the agent that inhibits IL-6 signaling is selected from the group consisting of a small molecule, an antibody, a peptide, a genome editing system, an antisense oligonucleotide, and an RNAi.

12) The method of claim 11, wherein the RNAi is a microRNA, an siRNA, or a shRNA.

13) The method of claim 11, wherein the antibody is a humanized antibody.

14) The method of claim 13, wherein the humanized antibody is tocilizumab.

15) The method of any of claim 11-14, wherein the agent targets IL-6 or IL-6 receptor (IL-6R).

16) The method of any of claim 1, wherein inhibiting IL-6 or TL-6R is inhibiting the expression level and/or activity of IL-6 or IL-6R.

17) The method of claim 16, wherein the expression level and/or activity of IL-6 or IL-6R is inhibited by at least 50%, at least 60%, at least 70%, at least 80%, at least 90%, or more as compared to an appropriate control.

18) The method of claim 1, wherein administration decreases circulation of a cell selected from the group consisting of: a IL-4⁺Foxp3⁺ (T_{H}2-like) T regulatory cell, a IL-17⁺Foxp3⁺ (T_{H}17-like) T regulatory cell, a T_{H}2 cell, and a T_{H}17 cell.

19) The method of claim 18, wherein circulation is decreased by at least 50%, at least 60%, at least 70%, at least 80%, at least 90%, or more as compared to an appropriate control.

20) The method of claim 1, wherein the agent is administered at a concentration of 8 mg/kg or 10 mg/kg.

21) The method of claim 1, further comprising administering at least a second asthma therapeutic.

22) A method of treating asthma, the method comprising administering to a subject in need thereof an effective amount of tocilizumab.

23) The method of claim 22, wherein the asthma is pediatric asthma.

24) The method of claim 22 or 23, wherein the asthma is non-atopic asthma.

25) The method of any of claims 22-24, wherein the asthma is severe, persistent asthma.

26) The method of claim 22, further comprising, prior to administration, diagnosing a subject with having asthma.

27) The method of any of claims 22-26, wherein the subject has a mutation in the TL4R gene.

28) The method of claim 27, wherein the subject is a homozygous for IL4R dominant allele.

29) The method of claim 27, wherein the subject is a homozygous for IL4R mutant allele.

30) The method of claim 22, further comprising, prior to administration, identifying a subject as having a mutation in the TL4R gene.

31) The method of claim 22, further comprising, prior to administration, receiving results that identify a subject as having a mutation in the TL4R gene.

32) The method of claim 22, wherein tocilizumab is administered at a concentration of 8 mg/kg or 10 mg/kg.

33) The method of claim any of claims 22-32, wherein tocilizumab is administered once every 2 weeks or once every 4 weeks.

34) The method of claim 22, wherein administration decreases circulation of a cell selected from the group consisting of: a IL-4⁺Foxp3⁺ (T_{H}2-like) T regulatory cell, a IL-17⁺Foxp3⁺ (T_{H}17-like) T regulatory cell, a T_{H}2 cell, and a T_{H}17 cell.

35) The method of claim 34, wherein circulation is decreased by at least 50%, at least 60%, at least 70%, at least 80%, at least 90%, or more as compared to an appropriate control.

36) The method of claim 22, further comprising administering at least a second asthma therapeutic.

37) A composition comprising an agent that inhibits IL-6 signaling.

38) The composition of claim 37, further comprising a pharmaceutically acceptable carrier.


SEQUENCE LISTING (4 sequences)

SEQ ID NO: 1 - DNA, 640 bp, Homo sapiens
tatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 2 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - DNA, 1407 bp, Homo sapiens
atgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatag

SEQ ID NO: 4 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR